SEQUENCE LISTING (59 sequences)

SEQ ID NO: 1 - protein, 6 aa, Artificial Sequence
  PAR2-AP
SLIGKV

SEQ ID NO: 2 - DNA, 21 bp, Artificial Sequence
  Human PAR1 F primer
ccattttgggaggatgaggag

SEQ ID NO: 3 - DNA, 23 bp, Artificial Sequence
  Human PAR1 R primer
aggatgaacacaacgatggccat

SEQ ID NO: 4 - DNA, 23 bp, Artificial Sequence
  Human PAR2 F primer
atggcacatcccacgtcactgga

SEQ ID NO: 5 - DNA, 24 bp, Artificial Sequence
  Human PAR2 R primer
gaaccagatgacagagaggaggtc

SEQ ID NO: 6 - DNA, 22 bp, Artificial Sequence
  Human PAR3 F primer
atgctaccatggggtacctgac

SEQ ID NO: 7 - DNA, 24 bp, Artificial Sequence
  Human PAR3 R primer
gttgccatagaagatgactgtggt

SEQ ID NO: 8 - DNA, 22 bp, Artificial Sequence
  Human PAR4 F primer
cctccaccatgctgctgatgaa

SEQ ID NO: 9 - DNA, 21 bp, Artificial Sequence
  Human PAR4 R primer
aggtctgccgctgcagtgtca

SEQ ID NO: 10 - DNA, 22 bp, Artificial Sequence
  Human Actin F primer
ggtcatcaccattggcaatgag

SEQ ID NO: 11 - DNA, 22 bp, Artificial Sequence
  Human Actin R primer
gatcttgatcttcattgtgctg

SEQ ID NO: 12 - DNA, 21 bp, Artificial Sequence
  Monkey PAR1 F primer
ccattttgggaggatgaggag

SEQ ID NO: 13 - DNA, 23 bp, Artificial Sequence
  Monkey PAR1 R primer
aggatgaacacaacgatggccat

SEQ ID NO: 14 - DNA, 23 bp, Artificial Sequence
  Monkey PAR2 F primer
atggcacatcccacgtcactgga

SEQ ID NO: 15 - DNA, 24 bp, Artificial Sequence
  Monkey PAR2 R primer
gaaccagatgacagagaggaggtc

SEQ ID NO: 16 - DNA, 22 bp, Artificial Sequence
  Monkey PAR3 F primer
atgctaccatggggtacctgac

SEQ ID NO: 17 - DNA, 24 bp, Artificial Sequence
  Monkey PAR3 R primer
gttgccatagaagatgactgtggt

SEQ ID NO: 18 - DNA, 22 bp, Artificial Sequence
  Monkey PAR4 F primer
cctccaccatgctgctgatgaa

SEQ ID NO: 19 - DNA, 21 bp, Artificial Sequence
  Monkey PAR4 R primer
aggtctgccgctgcagtgtca

SEQ ID NO: 20 - DNA, 21 bp, Artificial Sequence
  Monkey Actin F primer
ggcaccacaccttctacaatg

SEQ ID NO: 21 - DNA, 21 bp, Artificial Sequence
  Monkey Actin R primer
ggtccagacgcaggatggcat

SEQ ID NO: 22 - DNA, 21 bp, Artificial Sequence
  Hamster PAR1 F primer
cgccagccagaatctgagatg

SEQ ID NO: 23 - DNA, 21 bp, Artificial Sequence
  Hamster PAR1 R primer
cgaggggatgaagagcctcag

SEQ ID NO: 24 - DNA, 21 bp, Artificial Sequence
  Hamster PAR2 F primer
ggacgcaacggtaaaggaaga

SEQ ID NO: 25 - DNA, 21 bp, Artificial Sequence
  Hamster PAR2 R primer
cttcgtccggaaaaggaagac

SEQ ID NO: 26 - DNA, 21 bp, Artificial Sequence
  Hamster PAR3 F primer
cttctgccagccactttttgc

SEQ ID NO: 27 - DNA, 24 bp, Artificial Sequence
  Hamster PAR3 R primer
ggaacttctcaggtatcccatggt

SEQ ID NO: 28 - DNA, 21 bp, Artificial Sequence
  Hamster PAR4 F primer
gggaaattctgtgccaacgac

SEQ ID NO: 29 - DNA, 21 bp, Artificial Sequence
  Hamster PAR4 R primer
ggccaatagtaggtccgaaac

SEQ ID NO: 30 - DNA, 21 bp, Artificial Sequence
  Hamster Actin F primer
gtagccattcaggctgtgctg

SEQ ID NO: 31 - DNA, 21 bp, Artificial Sequence
  Hamster Actin R primer
atgcagcagtggccatctcct

SEQ ID NO: 32 - DNA, 48 bp, Artificial Sequence
  PAR2 F primer
atgtctgaattcgccaccatgcggagccccagcgcggcgtggctgctg

SEQ ID NO: 33 - DNA, 44 bp, Artificial Sequence
  PAR2 R primer
atgtctgcggccgctcaataggaggtcttaacagtggttgaact

SEQ ID NO: 34 - DNA, 912 bp, Artificial Sequence
  IgG-Fc PAR2 N-terminus with signal peptide
atgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactggcgcgccaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 35 - protein, 303 aa, Artificial Sequence
  IgG-Fc PAR2 N-terminus with signal peptide
MRSPSAAWLLGAAILLAASLSCSGTIQGTNRSSKGRSLIGKVDGTSHVTGKGVTVETVFSVDEFSASVLTGKLTTGAPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 843 bp, Artificial Sequence
  IgG-Fc PAR2 N-terminus without signal peptide
atgaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactggcgcgccaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 280 aa, Artificial Sequence
  IgG-Fc PAR2 N-terminus without signal peptide
MTIQGTNRSSKGRSLIGKVDGTSHVTGKGVTVETVFSVDEFSASVLTGKLTTGAPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 777 bp, Artificial Sequence
  IgG-Fc insulin signal peptide
atggccctgtggatgcgcctcctgcccctgctggcgctgctggccctctggggacctgacccagccgcagcctttgtgaaccaacacctgggcgcgccaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 39 - protein, 258 aa, Artificial Sequence
  IgG-Fc insulin signal peptide
MALWMRLLPLLALLALWGPDPAAAFVNQHLGAPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 789 bp, Artificial Sequence
  IgG-Fc insulin receptor signal peptide
atggccaccgggggccggcggggggcggcggccgcgccgctgctggtggcggtggccgcgctgctactgggcgccgcgggccacctgtaccccggagaggtgggcgcgccaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 262 aa, Artificial Sequence
  IgG-Fc insulin receptor signal peptide
MATGGRRGAAAAPLLVAVAALLLGAAGHLYPGEVGAPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 7 aa, Artificial Sequence
  N-terminal PAR2 sequence
TIQGTNR

SEQ ID NO: 43 - protein, 7 aa, Artificial Sequence
  N-terminal PAR2 sequence variant
TIQGTDR

SEQ ID NO: 44 - DNA, 1125 bp, Artificial Sequence
  PAR2deltaSP
atgaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 45 - protein, 374 aa, Artificial Sequence
  PAR2deltaSP
MTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 46 - DNA, 1212 bp, Artificial Sequence
  PAR2-INSP
atggccctgtggatgcgcctcctgcccctgctggcgctgctggccctctggggacctgacccagccgcagcctttgtgaaccaacacctgaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 47 - protein, 403 aa, Artificial Sequence
  PAR2-INSP
MALWMRLLPLLALLALWGPDPAAAFVNQHLTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 48 - DNA, 1224 bp, Artificial Sequence
  PAR2-IRSP
atggccaccgggggccggcggggggcggcggccgcgccgctgctggtggcggtggccgcgctgctactgggcgccgcgggccacctgtaccccggagaggtgaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 49 - protein, 407 aa, Artificial Sequence
  PAR2-IRSP
MATGGRRGAAAAPLLVAVAALLLGAAGHLYPGEVTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 50 - DNA, 1071 bp, Artificial Sequence
  PAR2deltaSPdeltaL
atggatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 51 - protein, 356 aa, Artificial Sequence
  PAR2deltaSPdeltaL
MDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 52 - DNA, 1125 bp, Artificial Sequence
  PAR2deltaSP(R36A)
atgaccatccaaggaaccaatagatcctctaaaggagcaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 53 - protein, 374 aa, Artificial Sequence
  PAR2deltaSP(R36A)
MTIQGTNRSSKGASLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 54 - DNA, 1194 bp, Artificial Sequence
  PAR2(R36A)
atgcggagccccagcgcggcgtggctgctgggggccgccatcctgctagcagcctctctctcctgcagtggcaccatccaaggaaccaatagatcctctaaaggagcaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 55 - protein, 397 aa, Artificial Sequence
  PAR2(R36A)
MRSPSAAWLLGAAILLAASLSCSGTIQGTNRSSKGASLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 56 - DNA, 1194 bp, Artificial Sequence
  PAR2 WT
atgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 57 - protein, 397 aa, Artificial Sequence
  PAR2 WT
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 58 - protein, 6 aa, Artificial Sequence
  PAR2 agonist
  MISC_FEATURE (6)..(6): Leucine with an amine group chemical modification
SLIGRL

SEQ ID NO: 59 - protein, 5 aa, Artificial Sequence
  PAR2 agonist
  MISC_FEATURE (1)..(1): Leucine with a 2-furoyl chemical modification
  MISC_FEATURE (5)..(5): Leucine with an amine group chemical modification
LIGRL

(19) United States
(12) Patent Application Publication
(10) Pub. No.: US 2022/0125898 A1
(43) Pub. Date: Apr. 28, 2022

(54) PAR2 MODULATION AND METHODS THEREOF

(71) Applicant: JANSSEN BIOTECH, INC., Horsham, PA (US)

(72) Inventors: Changlu LIU, San Diego, CA (US); Siquan SUN, San Diego, CA (US); Grace STEEMERS-LEE, San Diego, CA (US); Belinda LIU, San Diego, CA (US)

(73) Assignee: JANSSEN BIOTECH, INC., Horsham, PA (US)

(21) Appl. No.: 17/607,235

(22) PCT Filed: Apr. 30, 2020
(86) PCT No.: PCT/IB2020/054114

§ 371 Date: Oct. 28, 2021

Related U.S. Application Data
(60) Provisional application No. 62/842,869, filed on May 3, 2019.

(51) Int. Cl. A61K 38/57; C07K 14/47; A61K 31/137; A61K 38/06; G01N 33/50

(52) U.S. Cl. CPC A61K 38/57; C07K 14/47; G01N 2333/726; A61K 38/06; G01N 33/5041; A61K 31/137

(57) ABSTRACT

Provided herein are methods of identifying an agent that activates a protease-activated receptor 2 (PAR2)intracellularly. Also provided are isolated mutant PAR2 polypeptides, isolated polynucleotides encoding the mutant PAR2 polypeptides, vectors comprising the isolated polynucleotides, and host cells comprising the vectors.

### CROSS REFERENCE TO RELATED APPLICATIONS

This application claims priority to U.S. Provisional Application No. 62/842,869, filed on May 3, 2019, the disclosure of which is incorporated herein by reference in its entirety.

### FIELD OF THE INVENTION

This invention relates to the identification of methods of identifying agents that activate a protease-activated receptor 2 (PAR2) intracellularly. The invention also relates to isolated mutant PAR2 polypeptides, nucleic acids encoding the peptides, vectors comprising the nucleic acids, and host cells comprising the vectors.

### REFERENCE TO SEQUENCE LISTING SUBMITTED ELECTRONICALLY

This application contains a sequence listing, which is submitted electronically via EFS-Web as an ASCII formatted sequence listing with a file name “JBI6090WOPCT1SEQLIST.TXT” and a creation date of Apr. 15, 2020 and having a size of 57 kb. The sequence listing submitted via EFS-Web is part of the specification and is herein incorporated by reference in its entirety.

### BACKGROUND OF THE INVENTION

G-protein coupled receptors (GPCRs) are a class of 7 transmembrane domain cell surface receptors and consist of the largest receptor family in mammals and other organisms. They are involved in the signal transduction of almost every system in human physiology, including the sensory (visual, taste, olfactory), metabolic, endocrine, immune, and the nervous systems. Unlike many other cell surface receptors that have a classical signal peptide to lead the proteins to the cell surface, the majority of GPCRs (>90%) do not have a signal peptide (Schülein et al., 2011). In general, class B receptors such as the secretin receptor (Tam et. 2014), CRH receptors (Schulein et al., 2017), the Glucagon receptor (Zhang et al., 2017), and Glucagon-like peptide receptors (Huang et al., 2010) and the class C GPCRs, such as metabotropic glutamate receptors (Choi et al, 2011), GABA receptors (White et al., 1998), and adhesion GPCRs (Liebscher et al., 2014),which have relatively large N-terminal extracellular domains are more likely to have signal peptides than class A receptors (FIG. 1A). It is hypothesized that the presence of the signal peptide helps the large hydrophilic N-terminus to cross the plasma membrane. Most class A GPCRs do not have classical signal peptides. It is believed that the first transmembrane domain of these class A GPCRs serves as a signal anchor sequence to help these receptors translocate to the cell membrane after translation and assembly in the endoplasmic reticulum (ER) (Rutz et al., 2015).

Protease-activated receptors (PARs), including PAR1, PAR2, PAR3, and PAR4 belong to class A GPCR receptor sub-family (Macfarlane et al., 2001). Homology-wise, they are very closely related to cysteinyl leukotriene receptors (CYSLT), niacin receptors (GPR109), lactic acid receptor (GPR81), and the succinate receptor (GPR91). Unlike their closest neighbors (FIG. 1B), which do not possess a signal peptide, all PARs have a predicted signal peptide at their N-termini (FIG. 1C). Genomic analyses show, in contrast to their closest neighbors that are all encoded by single exon genes, PARs have an additional exon encoding only the signal peptides (FIG. 1C), suggesting that these signal peptides may play a specific role for PARs. As disclosed herein, PAR2 was utilized to study the importance of the signal peptide in PAR receptor function and localization.

### BRIEF SUMMARY OF THE INVENTION

In one general aspect, the invention relates to the identification of methods of identifying agents that activate a protease-activated receptor 2 (PAR2) intracellularly. The invention also relates to isolated mutant PAR2 polypeptides, nucleic acids encoding the peptides, vectors comprising the nucleic acids, and host cells comprising the vectors.

Provided herein are methods of identifying an agent that activates a protease-activated receptor intracellularly. The methods comprise (a) providing a cell expressing the protease activated receptor on a surface of the cell, wherein the protease activated receptor comprises a signal peptide sequence; (b) contacting the cell with an agent; (c) measuring a level of protease activated receptor on the surface of the cell, wherein a reduction in the level of protease activated receptor on the surface of the cell as compared to a control indicates that the agent is capable of activating the protease activated receptor intracellularly.

In certain embodiments, the methods of identifying an agent that activates a protease activated receptor intracellularly comprises (a) providing a cell expressing the protease activated receptor on a surface of the cell, wherein the protease activated receptor comprises a signal peptide sequence; (b) contacting the cell with an agent; (c) contacting the cell with a protease and/or a peptide ligand or small molecule; and (d) measuring a level of activation of the protease activated receptor upon contacting the cell with the protease and/or peptide ligand, wherein a reduction in the level of activation of the protease activated receptor as compared to a control indicates that the agent is capable of activating the protease activated receptor intracellularly.

In certain embodiments, the protease activated receptor is selected from the group consisting of protease-activated receptor 1 (PAR1), PAR2, PAR3, and PAR4.

Provided herein are methods of identifying an agent that activates a protease-activated receptor 2 (PAR2) intracellularly. The methods comprise (a) providing a cell expressing the PAR2 on a surface of the cell, wherein the PAR2 comprises a signal peptide sequence; (b) contacting the cell with an agent; (c) measuring a level of PAR2 on the surface of the cell, wherein a reduction in the level of PAR2 on the surface of the cell as compared to a control indicates that the agent is capable of activating PAR2 intracellularly.

In certain embodiments, the methods of identifying an agent that activates a protease-activated receptor 2 (PAR2) intracellularly comprises (a) providing a cell expressing the PAR2 on a surface of the cell, wherein the PAR2 comprises a signal peptide sequence; (b) contacting the cell with an agent; (c) contacting the cell with a protease and/or a peptide ligand or small molecule; and (d) measuring a level of activation of PAR2 upon contacting the cell with the protease and/or peptide ligand, wherein a reduction in the level of activation of PAR2 as compared to a control indicates that the agent is capable of activating PAR2 intracellularly.

In certain embodiments, the PAR1, PAR2, PAR3, or PAR4 is endogenously or exogenously expressed. In certain embodiments, endogenous PAR1, PAR2, PAR3, or PAR4 expression is substantially eliminated.

In certain embodiments, the cell is selected from the group consisting of a CHO-K1 cell, a COS-7 cell, and a HEK293 cell.

In certain embodiments, the agent is selected from the group consisting of a small molecule, a polypeptide, an antibody, a lipid, a polysaccharide, and a polynucleotide.

In certain embodiments, the control is a cell engineered to express a mutant protease activated receptor polypeptide, preferably wherein the mutant protease activated receptor polypeptide is a mutant PAR2 polypeptide. The mutant PAR2 polypeptide can, for example, comprise an amino acid sequence with at least 95% identity to SEQ ID NO:55.

In certain embodiments, the agent binds the signal peptide sequence of the PAR1, PAR2, PAR3, or PAR4 intracellularly to disrupt the signal peptide function. In certain embodiments, the agent binds an allosteric site on the PAR1, PAR2, PAR3, or PAR4, wherein binding of the agent to the allosteric site disrupts the signal peptide function.

In certain embodiments, the protease is selected from the group consisting of trypsin, tryptase, factor Xa, factor VIIa, matriptase/MT-serine protease 1, cysteine proteinase (RgpB), dust mite proteinase Der p3, dust mite proteinase Der p9, furin, and thrombin.

In certain embodiments, the peptide ligand can comprise SLIGKV (SEQ ID NO:1), SLIGRL-NH2 (SEQ ID NO:58), or 2-furoyl-LIGRL-NH2 (SEQ ID NO:59).

In certain embodiments, the small molecule can be GB110.

Also provided are isolated mutant PAR2 polypeptides comprising an amino acid sequence selected from the group consisting of SEQ ID NO:45, SEQ ID NO:51, SEQ ID NO:53, and SEQ ID NO:55.

Also provided are isolated polynucleotides encoding the mutant PAR2 polypeptides of the invention. Also provided are vectors comprising the isolated polynucleotides of the invention. Also provided are host cells comprising the vectors of the invention.

Also provided are methods of producing an isolated mutant PAR2 polypeptide. The methods comprise culturing the host cell of the invention under conditions suitable for the expression of the mutant PAR2 polypeptide and recovering the mutant PAR2 polypeptide from the cell or culture.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1C show PAR receptors are unique group of receptors in the Class A subfamily. FIG. 1A shows examples of GPCR subfamily members and signal peptide possession. The signal peptide regions in Class B and C are shown. FIG. 1B shows PAR receptors and their closest neighbors, grouped by sequence similarity. FIG. 1C shows the N-terminal amino acid sequences of PAR1-4. The signal peptides are shown in bold. Each PAR receptor is encoded by 2 exons. The protein regions coded by the first exons are underlined. The Arg (R) residues involved in receptor cleavage and activation are shown in bold.

FIGS. 2A-2D show PAR2 signal peptide behaves like a classical signal peptide.

FIG. 2A shows expression constructs for testing the roles of the signal peptide of PAR2 in leading IgG-Fc secretion. The N-terminus of PAR2 with its signal peptide (PAR2), the N-terminus of PAR2 without the signal peptide (PAR2ASP), the N-terminus of insulin (IN), and the N-terminus of insulin receptor (IR) are fused to the human IgG-Fc fragment respectively. The signal peptide regions of PAR2, insulin, and insulin receptor are highlighted and underlined. Human IgG-Fc fragment is highlighted. FIGS. 2B and 2C show detection of IgG-Fc expression in cells by immuno-fluorescent staining and ELISA. COS7 cells expressing various IgG-Fc fusion proteins as indicated were fixed, penetrated using detergent, and then detected or stained by FTIC-labeled fluorescent antibodies (FIG. 2B) or by ELISA (FIG. 2C). For ELISA, experiments were performed in quadruplicates and the results are shown in mean±sd. Statistical analysis (One-Way ANOVA) demonstrated that, compared with the control (NC), PAR2 (** p=0.0019), PAR2ASP (* p=0.0249), IN (** p=0.0024), and IR (** p=0.0038) were expressed at significant levels. FIG. 2D shows detection of IgG-Fc secretion into media by ELISA. Serum free conditioned medium from COS7 cells expressing various IgG-Fc fusion proteins with different N-termini, including PAR2 N-terminus (PAR2), PAR2 N-terminus without the signal peptide (PAR2ASP), the N-terminus of insulin (IN), and the N-terminus of insulin receptor (IR). Untransfected cells were used as the negative control (NC). Experiments were performed in quadruplicates and the results are shown in mean sd. Statistical analysis (One-Way ANOVA) showed that, compared with the control (NC), PAR2, IN, and IR all showed a great amount of secreted IgG-Fc protein (**** p <0.0001). All experiments were performed 3 times and very similar results were observed.

FIG. 3 shows the determination of the amino (N)-terminal sequence of PAR2 mature protein. The N-terminal extracellular region of PAR2 is fused to the N-terminus of IgG-Fc. The predicted signal peptide of PAR2 is shown and underlined. The IgG-Fc region is shown. The potential N-linked glycosylation site, NRS, is underlined. The protein was expressed in COS7 cells and affinity purified. The N-terminus of the purified protein was determined by MS sequencing after trypsin digestion. Two sequences were observed: TIQGTNR (SEQ ID NO:42) and TIQGTDR (SEQ ID NO:43) representing unglycosylated and glycosylated PAR2 N-termini.

FIGS. 4A-4F show CHO-K1, COS7, and HEK293 cells express PAR1 and PAR2 receptors. FIG. 4A demonstrates that CHO-K1, COS7, and HEK293 cells naturally express high levels of PAR1 and PAR2 mRNA but express little or no PAR3 and PAR4 mRNA. qPCR analysis was used to quantify the mRNA expression. Specific primers for each of PAR1, PAR2, PAR3, and PAR4, were used to quantify the respective mRNA expression using cDNA made from each cell line as the template. β-actin primers were used to quantify β-actin mRNA expression as the internal control. The relative mRNA expression of PAR1, PAR2, PAR3, and PAR4 were first normalized using β-actin expression, and then normalized using the PAR1 expression level in CHO-K1 cells, which is arbitrarily set as 100%. The relative expressions of other genes were represented as a percentage of PAR1 mRNA level in CHO-K1 cells. The results shown are mean±sd (n=3). Statistical analysis (One-Way ANOVA) showed that compared with the mRNA expression of PAR4, which is undetectable in these cells, CHO cells expressed high levels of mRNAs for PAR1 (** p=0.0037), PAR2 (* p=0.023), and PAR3 (* p=0.035); COS7 and HEK293 cells express high level of mRNAs for PAR1 (** p=0.0029, * p=0.032, respectively) and PAR2 (** p=0.0013, ** p=0.0027, respectively) without expressing detectable PAR3 and PAR4 mRNAs. FIGS. 4B, 4C, and 4D demonstrated that CHO-K1, COS7, and HEK293 cells naturally expressed PAR1 and PAR2 receptors and responded to thrombin (PAR1 ligand) and trypsin (PAR2 ligand) stimulations. FLIPR assays were used to measure receptor activation as indicated by intracellular Ca²⁺ mobilization. Relative fluorescent units (RFU) were the readout for fluorescent intensities for Ca²⁺ mobilization signals. Various concentrations of thrombin or trypsin were used as the ligands to activate the receptors. The assays were performed in triplicate at each data point and mean±sd are shown. FIG. 4E shows sequencing analysis of the genomic DNA from par1 and par2 knock out HEK293 cells. The results show that a 270 bp deletion in par1 gene and a 347 bp deletion in part gene have been achieved. The deletions removed the coding regions from TM2 to TM3 for both PAR1 and PAR2 proteins. The vertical lines indicate the deletion sites. FIG. 4F shows the characterization of par1 and par2 knock-out HEK293 cells. FLIPR assays were used to characterize receptor activation as indicated. Wild type HEK293 cells were used as the positive control. The assays were performed in triplicate at each data point and mean±sd are shown.

FIGS. 5A-5C demonstrate that the signal peptide is important for functional expression of PAR2. FIG. 5A shows a schematic diagram showing the modifications to PAR2 receptor. The N-terminal extracellular sequences of various PAR2 mutants are shown. Human PAR2 wild type (PAR2) (SEQ ID NO:57), PAR2 with the signal peptide deleted (PAR2ΔSP) (SEQ ID NO:45), PAR2 with an insulin signal peptide (PAR2-INSP) (SEQ ID NO:47) and an insulin receptor signal peptide (PAR2-IRSP) (SEQ ID NO:49). The native signal peptide of PAR2, the insulin signal peptide, and the insulin receptor signal peptide are shown. The tether ligand sequence of PAR2 (SLIGKV) (SEQ ID NO:1) is underlined. FIGS. 5B and 5C show the characterization of PAR2 mutants in FLIPR assay using trypsin or the synthetic PAR2 agonist peptide (PAR2-AP) (SEQ ID NO:1) as the ligands. Expression constructs for PAR2 wild type receptor and various modifications were cloned into pcDNA3.1 and transiently expressed in HEK293 cells with par1 and par2 knocked-out. Various concentrations of trypsin (FIG. 5B) or PAR-AP (SEQ ID NO:1) (FIG. 5C) were added to stimulate the intracellular Ca²⁺ mobilization. Relative fluorescent intensity units (RFU) are shown. The experiments were performed in triplicate at each data point and the results shown are mean±sd. HEK293 cells with par1 and par2 genes knocked-out were used as the host cells for recombinant expression of various PAR2 receptors. Untransfected cells were used as the negative controls (NC).

FIGS. 6A-6C shows that further deletion of the tethered ligand rescues the functional expression of PAR2 without the signal peptide. FIG. 6A shows the schematic diagram showing the modifications to PAR2 receptor. The N-terminal extracellular sequences of various PAR2 mutants are shown. Human PAR2 wild type (PAR2) (SEQ ID NO:57), PAR2 with the signal peptide deleted (PAR2ΔSP) (SEQ ID NO:45), PAR2 with the signal peptide deletion and with further deletion to the tether ligand region (PAR2ΔSPΔL) (SEQ ID NO:51). The signal peptide of PAR2 is shown. The tether ligand sequence of PAR2 (SLIGKV) (SEQ ID NO:1) is underlined. FIGS. 6B and 6C show the characterization of mutant PAR2 receptors using FLIPR assays. Various PAR2 expression constructs were transiently expressed in HEK293 with par1 and par2 knocked-out. Trypsin (FIG. 6B) or the synthetic agonist peptide PAR2 ligand (PAR2-AP) (SEQ ID NO:1) (FIG. 6C) were used as the ligand to stimulate receptor activation. HEK293 cells with par1 and par2 genes knocked-out were used as the host cells for recombinant expression of various PAR2 receptors. Untransfected cells were used as the negative controls (NC). The experiments were performed in triplicate at each data point and the results shown are mean±sd.

FIGS. 7A-7C show that the Arg³⁶ to Ala mutation helps the functional expression of PAR2 without a signal peptide. FIG. 7A shows the schematic diagram showing the modifications/mutations to PAR2 receptor. The N-terminal extracellular sequences of various PAR2 mutants are shown. PAR2 wild type (PAR2) (SEQ ID NO:57), PAR2 with an Arg36Ala mutation (PAR2(R36A)) (SEQ ID NO:55), PAR2 with the signal peptide deleted (PAR2ΔSP) (SEQ ID NO:45), PAR2 with the signal peptide deletion and with an Arg36Ala mutation (PAR2ΔSP(R36A)) (SEQ ID NO:53) were used for characterizations. The signal peptide of PAR2 is shown. The tether ligand sequence of PAR2 (SLIGKV) (SEQ ID NO:1) is underlined. The Ala residue substituted for Arg36, which is involved in trypsin cleavage/activation of PAR2, is highlighted. The mutant receptors were characterized in FLIPR assays using either trypsin (FIG. 7B) or PAR2-AP (FIG. 7C) as ligands. HEK293 cells with par1 and par2 genes knocked-out were used as the host cells for recombinant expression. Untransfected cells were used as the negative controls (NC). The experiments were performed in triplicate at each data point and the results shown are mean±sd.

FIGS. 8A-8E show that a serine protease inhibitor cocktail increases the functional expression of PAR2 without the signal peptide. HEK293 cells with par1 and par2 knocked out were used for the transient expression of various PAR2 proteins. Treatment with protease inhibitor cocktail (PI) lowered the Emax values for all receptors with similar degrees. Protease treated samples showed about 80% response in Emax values compared with those of untreated cells. For comparison of the EC₅₀ values between samples treated and untreated with the protease inhibitor cocktails, the results were normalized using their Emax values and the data were expressed as the percentages of the Emax. The experiments were performed in triplicate at each data point and the results shown are mean±sd.

FIG. 9 shows cell surface and total protein expression of PAR2 wild type and mutants. HEK293 cells with par1 and par2 knocked-out were used for the transient expression of various PAR2 proteins. PAR2 peptide ligand, PAR2-AP and protease inhibitor cocktails (PI) were used for treatments. Medium was used as the control treatment. ELISA with or without cell penetrating reagent was used to measure the total cell surface and protein expression. The experiments were performed in triplicate at each data point and the results shown are mean±sd. Statistical analysis (One-Way ANOVA) showed that, for both cell surface and total proteins, compared with PAR2, PAR2ΔSP, PAR2ΔSP(R36A), and PAR2ΔSPΔL have lower protein expression (**** p<0.0001). Compared with PAR2ΔSP, PAR2ΔSP(R36A) has much higher protein expression ($$$$ p<0.0001). Except for PAR2ΔSP, PAR2-AP decreased protein expression for all others (####p<0.0001). Protease inhibitor cocktails (PI) only increased the protein expression for PAR2ΔSP (++++p<0.0001) and did not affect the protein expressions for others. The experiments were performed 3 times and very similar results were observed.

FIGS. 10A-10C show that the Arg36Ala mutation and protease inhibitors increase the cell surface expression of PAR2-GFP without a signal peptide. FIG. 10A shows a schematic presentation of various PAR2-GFP fusion protein expression constructs. FIG. 10B shows the expression levels of various PAR2-GFP proteins with treatments of PAR2-AP, or protease inhibitors. Various PAR2-GFP expression constructs were transiently expressed in HEK293 cells with par1 and part knocked-out. The transfected cells were treated either with medium (medium), peptide agonist (PAR2-AP), or a protease inhibitor cocktails (PI), and the fluorescent intensities of the cells expressing the PAR2-GFP fusion proteins were measured. Assays were performed in quadruplicate at each data point and the results shown are mean±sd. Statistical analysis (One-Way ANOVA) showed that compared with PAR2, PAR2ΔSP and PAR2ΔSP(R36A) have lower protein expression (**** p<0.0001). Compared with PAR2ΔSP, PAR2ΔSP(R36A) has much higher protein expression ($$$$ p<0.0001). Except for PAR2ΔSP, PAR2-AP decreased protein expressions for all others (####p<0.0001). Protease inhibitor cocktails (PI) only increased the protein expression for PAR2ΔSP (++++p<0.0001) and did not affect the protein expression for others. FIG. 10C shows fluorescent images from confocal microscope showing the cellular distributions of various PAR2-GFP fusion proteins under the treatments of PAR2-AP or protease inhibitors. Untransfected cells were used as the negative control (NC). The fluorescent intensities are automatically adjusted for better viewing of the protein cellular distributions.

FIG. 11 shows a schematic diagram showing the proposed role of PAR2 signal peptide in protecting PAR2 from protease cleavage before reaching the plasma membrane. Without the signal peptide, the protease activation site of PAR2 is susceptible to protease cleavage in ER and Golgi, leading to PAR2 activation before reaching the cell surface and subsequent translocation to lysosome for degradation. With the signal peptide, PAR2 is bound by the signal peptide related translocon complex and segregated/protected from the cleavage by ER/Golgi proteases, allowing the receptor to reach the plasma membrane for sensing the extracellular trypsin activation. The signal peptide of PAR2 at the N-terminus is shown. The star at the N-terminus of PAR2 represents the cleavage/activation site (Arg36) by trypsin.

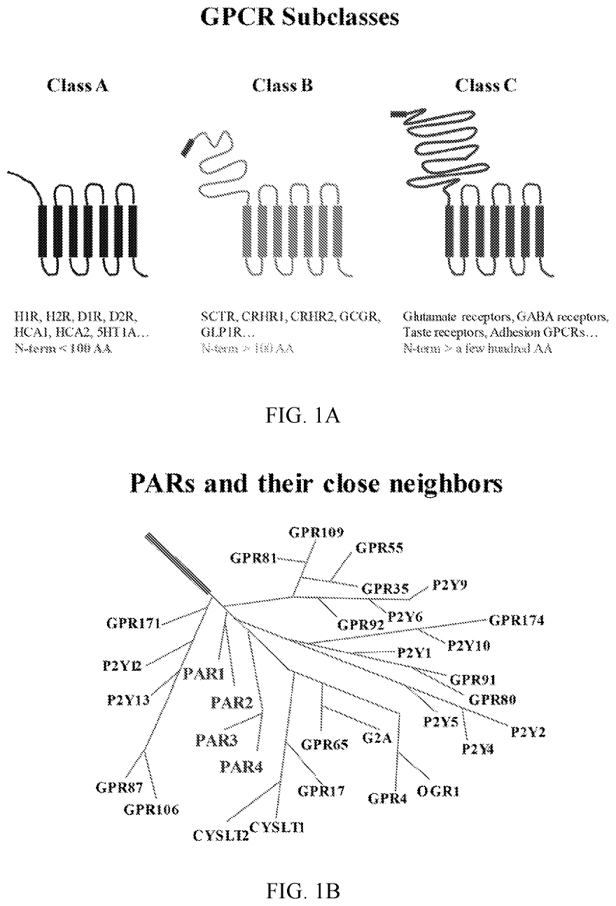

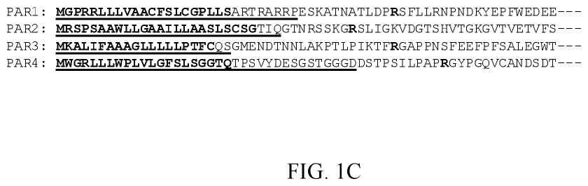

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., PAR2 polypeptides and PAR2 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the terms “peptide,” “polypeptide,” or “protein” can refer to a molecule comprised of amino acids and can be recognized as a protein by those of skill in the art. The convention one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein the term “PAR2” refers to the protease activated receptor 2 protein, which is a G-protein coupled receptor (GPCR). PAR2, along with family members PAR1, PAR3, and PAR4, is a member of the class A GPCR receptor sub-family. The PAR1, PAR2, PAR3, and PAR4 proteins have a predicted signal peptide, which is encoded by an additional exon in genes encoding PAR1 (F2R), PAR2 (F2RL1), PAR3 (F2RL2), and PAR4 (F2RL3).

As used herein the term “activation” refers to when an agonist binds a receptor (e.g., PAR2), which results in a signal cascade to the downstream pathways of the receptor. By way of an example, activation of PAR2 by an agent, as described herein, results in the activation of pathways that increases Ca′ intracellular influx, increases GTPγS binding (e.g., in increase in binding of G-protein to non-hydrolysable GTP analog GTPγS), increases β-arrestin recruitment (e.g., an increase in recruitment of β-arrestin to GPCR), increases cyclic AMP inhibition, and increases inositol phosphate-1 (IP) production.

As used herein the term “modulation” refers to a change in the level of activation of the receptor (e.g., PAR2). By way of an example, an agent can modulate the level of activation by decreasing the level of PAR2 activation (e.g., reducing Ca′ intracellular influx, reducing GTPγS binding, reducing β-arrestin recruitment, reducing cyclic AMP inhibition, and reducing IP production). An agent that decreases the level of PAR2 activation is an inhibitor of PAR2 activation (e.g., an antagonist). By way of another example, an agent can modulate the level of activation by increasing the level of PAR2 activation (e.g., increasing Ca²⁺ intracellular influx, increasing GTPγS binding, increasing β-arrestin recruitment, increasing cyclic AMP inhibition, and increasing IP production). An agent that increases the level of PAR2 activation is an enhancer of PAR2 activation (e.g., an agonist).

Methods of Identifying Agents that Increase Intracellular Protease Activated Receptor (e.g., PAR2) Activation

Provided herein are methods of identifying an agent that activates a protease-activated receptor intracellularly. The methods comprise (a) providing a cell expressing the protease activated receptor on a surface of the cell, wherein the protease activated receptor comprises a signal peptide sequence; (b) contacting the cell with an agent; (c) measuring a level of protease activated receptor on the surface of the cell, wherein a reduction in the level of protease activated receptor on the surface of the cell as compared to a control indicates that the agent is capable of activating the protease activated receptor intracellularly.

In certain embodiments, the methods of identifying an agent that activates a protease activated receptor intracellularly comprises (a) providing a cell expressing the protease activated receptor on a surface of the cell, wherein the protease activated receptor comprises a signal peptide sequence; (b) contacting the cell with an agent; (c) contacting the cell with a protease and/or a peptide ligand or small molecule; and (d) measuring a level of activation of the protease activated receptor upon contacting the cell with the protease and/or peptide ligand, wherein a reduction in the level of activation of the protease activated receptor as compared to a control indicates that the agent is capable of activating the protease activated receptor intracellularly.

In certain embodiments, the protease activated receptor is selected from the group consisting of protease-activated receptor 1 (PAR1), PAR2, PAR3, and PAR4. Provided herein are methods of identifying an agent that activates a protease-activated receptor 2 (PAR2) intracellularly. The methods comprise (a) providing a cell expressing the PAR2 on a surface of the cell, wherein the PAR2 comprises a signal peptide sequence; (b) contacting the cell with an agent; (c) measuring a level of PAR2 on the surface of the cell, wherein a reduction in the level of PAR2 on the surface of the cell as compared to a control indicates that the agent is capable of activating PAR2 intracellularly.

In certain embodiments, the methods of identifying an agent that activates a protease-activated receptor 2 (PAR2) intracellularly comprises (a) providing a cell expressing the PAR2 on a surface of the cell, wherein the PAR2 comprises a signal peptide sequence; (b) contacting the cell with an agent; (c) contacting the cell with a protease and/or a peptide ligand or small molecule; and (d) measuring a level of activation of PAR2 upon contacting the cell with the protease and/or peptide ligand, wherein a reduction in the level of activation of PAR2 as compared to a control indicates that the agent is capable of activating PAR2 intracellularly.

Determining a level of PAR1, PAR2, PAR3, or PAR4 in a cell can be done using methods known in the art and described below. When determining if an agent is capable of intracellularly activating PAR1, PAR2, PAR3, or PAR4, a level of PAR1, PAR2, PAR3, or PAR4 on the surface of the cell can be determined. The level of PAR1, PAR2, PAR3, or PAR4 on the surface of a cell contacted with the agent can be compared to the level of PAR1, PAR2, PAR3, or PAR4 on the surface of a control cell. In certain embodiments, the control cell is not contacted with an agent. In certain embodiments, the control cell is engineered to express a mutant protease activated receptor polypeptide, preferably wherein the mutant protease activated receptor is a mutant PAR2 polypeptide (e.g., a cell expressing a PAR2 polypeptide with an amino acid sequence with at least 95% identity to the amino acid sequence as set forth in SEQ ID NO: 55).

Determining a level of activation of protease activated receptor (e.g., PAR2) in a cell can be done using methods known in the art and described below. Determining a level of activation of protease activated receptor (e.g., PAR2) can be accomplished by determining a change in the intracellular Ca²⁺ mobilization, cyclic AMP inhibition, (3-arrestin recruitment, GTPγS binding, and/or IP production. When determining if an agent is capable of intracellularly activating a protease activated receptor (e.g., PAR2), a level of protease activated receptor (e.g., PAR2) activation can be determined. The level of protease activated receptor (e.g., PAR2) activation in a cell contacted with an agent can be compared to the level of protease activated receptor (e.g., PAR2) activation of a control cell. In certain embodiments, the control cell is not contacted with an agent. In certain embodiments, the control cell is engineered to express a mutant protease activated receptor (e.g., PAR2) polypeptide (e.g., a cell expressing a PAR2 polypeptide with an amino acid sequence with at least 95% identity to the amino acid sequence as set forth in SEQ ID NO:55).

Determining a level of activation of PAR2 can be accomplished by determining a change in the intracellular Ca²⁺ influx, cyclic AMP inhibition, β-arrestin recruitment, GTPγS binding, and/or inositol phosphate-1 (IP) production. An increase in intracellular PAR2 activation can lead to an increase in intracellular Ca²⁺ influx, an increase in cyclic AMP inhibition, an increase in β-arrestin recruitment, an increase in GTPγS binding, and an increase in IP production. A decrease in intracellular PAR2 activation can lead to a decrease in intracellular Ca²⁺ influx, a decrease in cyclic AMP inhibition, a decrease in β-arrestin recruitment, a decrease in GTPγS binding, and a decrease in IP production. Assays to determine changes in intracellular Ca²⁺ influx, cyclic AMP inhibition, β-arrestin recruitment, GTPγS binding, and IP production are known in the art, see, e.g., Liu et al., Mol. Pharmacol. 88:911-25 (2015); Liu et al., J. Biol. Chem. 284:2811-22 (2009); Liu et al., Nature 475 (7357):519-23 (2011); and Trinquet et al., Expert Opin. Drug. Discov. 6:981-94 (2011).

In certain embodiments, the PAR1, PAR2, PAR3, or PAR4 is endogenously expressed. Cells endogenously expressing PAR1, PAR2, PAR3, or PAR4 are known in the art and can include, but are not limited to CHO-K1 cells, COS-7 cells, and HEK293 cells. In certain embodiments, endogenous PAR1, PAR2, PAR3, or PAR4 expression is substantially eliminated. Endogenous PAR1, PAR2, PAR3, or PAR4 expression can be eliminated by knocking out the nucleotide sequence encoding PAR1, PAR2, PAR3, or PAR4 within the cell using methods known in the art for knocking out nucleotide sequences (e.g., homologous recombination, targeted deletion, etc.). Endogenous PAR1, PAR2, PAR3, or PAR4 expression can be eliminated by knocking down mRNA expression of PAR1, PAR2, PAR3, or PAR4 through RNAi technologies (e.g., short interfering RNAs and/or stable expression of a construct designed to produce miRNAs or short interfering RNAs capable of knocking down PAR1, PAR2, PAR3, or PAR4 mRNA expression).

In certain embodiments, the agent is selected from the group consisting of a small molecule, a polypeptide, an antibody, a lipid, a polysaccharide, and a polynucleotide. Agents can be identified from chemical libraries, natural product libraries, antibody libraries, peptide libraries, polysaccharide libraries, and polynucleotide libraries.

In certain embodiments, the agent binds the signal peptide sequence of the PAR1, PAR2, PAR3, or PAR4 intracellularly to disrupt the signal peptide function. Disruption of the signal peptide function can lead to reduced expression of the PAR1, PAR2, PAR3, or PAR4 in the cell. The reduced expression of the PAR1, PAR2, PAR3, or PAR4 in the cell can, for example, be due to cleavage of PAR1, PAR2, PAR3, or PAR4 by intracellular proteases (e.g., trypsin). Thus, binding of the agent to the signal peptide sequence of the PAR1, PAR2, PAR3, or PAR4 can lead to the disruption of the signal peptide function, which can result in a reduced level of PAR1, PAR2, PAR3, or PAR4 on the surface of the cell and/or a reduced level of PAR1, PAR2, PAR3, or PAR4 activation in the cell.

In certain embodiments, the agent binds an allosteric site on the PAR1, PAR2, PAR3, or PAR4, wherein binding of the agent to the allosteric site disrupts the signal peptide function. Binding of an agent to an allosteric site on the PAR1, PAR2, PAR3, or PAR4, can, for example, lead to a change in the structure of the PAR1, PAR2, PAR3, or PAR4 that can lead to a disruption of the signal peptide function. Disruption of the signal peptide function can lead to reduced expression of the PAR1, PAR2, PAR3, or PAR4 in the cell. Alternatively, disruption of the signal peptide function can lead to a reduced activation of the PAR1, PAR2, PAR3, or PAR4 in the cell, as the change in structure of the PAR1, PAR2, PAR3, or PAR4 could lead to reduced accessibility by the protease that activates the PAR1, PAR2, PAR3, or PAR4. Thus, binding of the agent to an allosteric site on the PAR1, PAR2, PAR3, or PAR4 can result in a reduced level of PAR1, PAR2, PAR3, or PAR4 on the surface of the cell and/or a reduced level of PAR1, PAR2, PAR3, or PAR4 activation in the cell.

In certain embodiments, the protease is selected from the group consisting of trypsin, tryptase, factor Xa TF, factor VIIa, matriptase/MT-serine protease 1, cysteine proteinase (RgpB), dust mite proteinase Der p3, dust mite proteinase Der p9, furin, and thrombin. Typsin can, for example, include, but is not limited to, trypsin-2, trypsin-3, trypsin IV, and trypsin (T1426)a.

In certain embodiments, the peptide ligand comprises SLIGKV (SEQ ID NO:1), SLIGRL-NH2 (SEQ ID NO:58), or 2-furoyl-LIGRL-NH2 (SEQ ID NO:59). Peptide ligands of PAR2 are known in the art, see, e.g., Kanke et al., Br. J. Pharmacol. 145:255-263 (2005).

In certain embodiments, the small molecule is GB110. Small molecule agonists of PAR2 are known in the art, see, e.g., Barry et al., J. Med. Chem. 53:7428-40 (2010).

Mutant PAR2 Polypeptides, Polynucleotides, and Cells Comprising the Same

In a general aspect, the invention relates to isolated mutant PAR2 polypeptides. The isolated mutant polypeptides can, for example comprise a deletion of the signal peptide, a deletion of the tethered ligand, a deletion of the signal peptide and the tethered ligand, a substitution of a protease cleavage site (e.g., Arg36 of SEQ ID NO:57). In certain embodiments, the isolated mutant PAR 2 polypeptides comprise an amino acid sequence selected from the group consisting of SEQ ID NO:45, SEQ ID NO:51, SEQ ID NO:53, and SEQ ID NO:55.

In certain embodiments, the isolated mutant PAR2 polypeptide comprises an amino acid sequence with at least 85% identity to the amino acid sequence set forth in SEQ ID NO:57, more preferably at least 90% identity with the amino acid sequence set forth in SEQ ID NO:57, still more preferably at least 95% identity with the amino acid sequence set forth in SEQ ID NO: 57, still more preferably at least 98% identity with the amino acid sequence set forth in SEQ ID NO: 57, most preferably at least 99% identity with the amino acid sequence set forth in SEQ ID NO: 57. In certain embodiments, the isolated mutant PAR2 polypeptide comprises an amino acid sequence with at least 85%, 86%, 87%, 88%, 89%, 90%, 91%, 92%, 93%, 94%, 95%, 96%, 97%, 98%, or 99% identity with the amino acid sequence set forth in SEQ ID NO:57.

In another general aspect, the invention relates to an isolated polynucleotide encoding the mutant PAR2 polypeptides of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding the mutant PAR2 polypeptides of the invention can be altered without changing the amino acid sequences of the proteins.

In another general aspect, the invention relates to a vector comprising an isolated polynucleotide encoding a mutant PAR2 of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of a fusion peptide in the cell. Conventional cloning techniques or artificial gene synthesis can be used to generate a recombinant expression vector according to embodiments of the invention.

In another general aspect, the invention relates to a host cell comprising an isolated polynucleotide encoding a mutant PAR2 polypeptide of the invention or a vector comprising an isolated polynucleotide encoding a mutant PAR2 polypeptide of the invention. Any host cell known to those skilled in the art in view of the present disclosure can be used for recombinant expression of mutant polypeptides of the invention. In some embodiments, the host cells are E. coli TG1 or BL21 cells, CHO-DG44 or CHO-1U cells or HEK293 cells. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a mutant PAR2 polypeptide of the invention. The methods comprise culturing a host cell comprising an isolated polynucleotide encoding the mutant PAR2 polypeptide of the invention under conditions suitable for the expression of the mutant PAR2 polypeptide and recovering the mutant PAR2 polypeptide from the cell or culture (e.g., from the supernatant). Expressed mutant PAR2 polypeptides can be harvested from the cells and purified according to conventional techniques known in the art and as described herein.

### Embodiments

This invention provides the following non-limiting embodiments.

Embodiment 1 is a method of identifying an agent that activates a protease activated receptor intracellularly, the method comprising:
a. providing a cell expressing the protease activated receptor on a surface of the cell, wherein the protease activated receptor comprises a signal peptide sequence;
b. contacting the cell with an agent;
c. measuring a level of protease activated receptor on the surface of the cell, wherein a reduction in the level of protease activated receptor on the surface of the cell as compared to a control indicates that the agent is capable of activating the protease activated receptor intracellularly.

Embodiment 2 is the method of embodiment 1, wherein the protease activated receptor is selected from the group consisting of protease-activated receptor 1 (PAR1), PAR2, PAR3, and PAR4.

Embodiment 3 is the method of embodiment 2 or 3, wherein PAR1, PAR2, PAR3, or PAR4 is endogenously or exogenously expressed.

Embodiment 4 is the method of embodiment 3, wherein PAR1, PAR2, PAR3, or PAR4 is exogenously expressed.

Embodiment 5 is the method of embodiment 4, wherein endogenous PAR1, PAR2, PAR3, or PAR4 expression is substantially eliminated.

Embodiment 6 is the method of any one of embodiments 1-5, wherein the cell is selected from the group consisting of a CHO-K1 cell, a COS-7 cell, and a HEK293 cell.

Embodiment 7 is the method of any one of embodiments 1-6, wherein the agent is selected from the group consisting of a small molecule, a polypeptide, an antibody, a lipid, a polysaccharide, and a polynucleotide.

Embodiment 8 is the method of any one of embodiments 1-7, wherein the control is a cell engineered to express a mutant protease activated receptor polypeptide, preferably wherein the mutant protease activated receptor polypeptide is a mutant PAR2 polypeptide.

Embodiment 9 is the method of embodiment 8, wherein the mutant PAR2 polypeptide comprises an amino acid sequence with at least 95% identity to SEQ ID NO:55.

Embodiment 10 is the method of any one of embodiments 1-9, wherein the agent binds the signal peptide sequence of the PAR1, PAR2, PAR3, or PAR4 intracellularly to disrupt the signal peptide function.

Embodiment 11 is the method of any one of embodiments 1-10, wherein the agent binds an allosteric site on the PAR1, PAR2, PAR3, or PAR4, wherein binding of the agent to the allosteric site disrupts the signal peptide function.

Embodiment 12 is a method of identifying an agent that activates a protease-activated receptor 2 (PAR2) intracellularly, the method comprising:
a. providing a cell expressing the PAR2 on a surface of the cell, wherein the PAR2 comprises a signal peptide sequence;
b. contacting the cell with an agent;
c. measuring a level of PAR2 on the surface of the cell, wherein a reduction in the level of PAR2 on the surface of the cell as compared to a control indicates that the agent is capable of activating PAR2 intracellularly.

Embodiment 13 is the method of embodiment 12, wherein PAR2 is endogenously or exogenously expressed.

Embodiment 14 is the method of embodiment 13, wherein PAR2 is exogenously expressed.

Embodiment 15 is the method of embodiment 14, wherein endogenous PAR2 expression is substantially eliminated.

Embodiment 16 is the method of any one of embodiments 12-15, wherein the cell is selected from the group consisting of a CHO-K1 cell, a COS-7 cell, and a HEK293 cell.

Embodiment 17 is the method of any one of embodiments 12-16, wherein the agent is selected from the group consisting of a small molecule, a polypeptide, an antibody, a lipid, a polysaccharide, and a polynucleotide.

Embodiment 18 is the method of any one of embodiments 12-17, wherein the control is a cell engineered to express a mutant PAR2 polypeptide.

Embodiment 19 is the method of embodiment 18, wherein the mutant PAR2 polypeptide comprises an amino acid sequence with at least 95% identity to SEQ ID NO:55.

Embodiment 20 is the method of any one of embodiments 12-19, wherein the agent binds the signal peptide sequence of the PAR2 intracellularly to disrupt the signal peptide function.

Embodiment 21 is the method of any one of embodiments 12-20, wherein the agent binds an allosteric site on the PAR2, wherein binding of the agent to the allosteric site disrupts the signal peptide function.

Embodiment 22 is a method of identifying an agent that activates a protease-activated receptor 2 (PAR2) intracellularly, the method comprising:
a. providing a cell expressing the PAR2 on a surface of the cell, wherein the PAR2 comprises a signal peptide sequence;
b. contacting the cell with an agent;
c. contacting the cell with a protease and/or a peptide ligand or small molecule;
d. measuring a level of activation of PAR2 upon contacting the cell with the protease and/or peptide ligand, wherein a reduction in the level of activation of PAR2 as compared to a control indicates that the agent is capable of activating PAR2 intracellularly.

Embodiment 23 is the method of embodiment 22, wherein PAR2 is endogenously or exogenously expressed.

Embodiment 24 is the method of embodiment 23, wherein PAR2 is exogenously expressed.

Embodiment 25 is the method of embodiment 24, wherein endogenous PAR2 expression is substantially eliminated.

Embodiment 26 is the method of any one of embodiments 22-25, wherein the cell is selected from the group consisting of a CHO-K1 cell, a COS-7 cell, and a HEK293 cell.

Embodiment 27 is the method of any one of embodiments 22-26, wherein the agent is selected from the group consisting of a small molecule, a polypeptide, an antibody, a lipid, a polysaccharide, and a polynucleotide.

Embodiment 28 is the method of any one of embodiments 22-27, wherein the control is a cell engineered to express a mutant PAR2 polypeptide.

Embodiment 29 is the method of embodiment 28, wherein the mutant PAR2 polypeptide comprises an amino acid sequence with at least 95% identity to SEQ ID NO:55.

Embodiment 30 is the method of any one of embodiments 22-29, wherein the agent binds the signal peptide sequence of the PAR2 intracellularly to disrupt the signal peptide function.

Embodiment 31 is the method of any one of embodiments 22-30, wherein the agent binds an allosteric site on the PAR2, wherein binding of the agent to the allosteric site disrupts the signal peptide function.

Embodiment 32 is the method of any one of embodiments 22-31, wherein the protease is selected from the group consisting of trypsin, tryptase, factor Xa TF, factor Vila, matriptase/MT-serine protease 1, cysteine proteinase (RgpB), dust mite proteinase Der p3, dust mite proteinase Der p9, furin, and thrombin.

Embodiment 33 is the method of any one of embodiments 22-32, wherein the peptide ligand comprises SLIGKV (SEQ ID NO:1), SLIGRL-NH2 (SEQ ID NO:58), or 2-furoyl-LIGRL-NH2 (SEQ ID NO:59).

Embodiment 34 is the method of any one of embodiments 22-33, wherein the small molecule is GB110.

Embodiment 35 is a method of identifying an agent that activates a protease-activated receptor intracellularly, the method comprising:
a. providing a cell expressing the protease activated receptor on a surface of the cell, wherein the protease activated receptor comprises a signal peptide sequence;
b. contacting the cell with an agent;
c. contacting the cell with a protease and/or a peptide ligand or small molecule;
d. measuring a level of activation of protease activated receptor upon contacting the cell with the protease and/or peptide ligand, wherein a reduction in the level of activation of protease activated receptor as compared to a control indicates that the agent is capable of activating protease activated receptor intracellularly.

Embodiment 36 is the method of embodiment 35, wherein the protease activated receptor is selected from the group consisting of protease activated receptor 1 (PAR1), PAR2, PAR3, and PAR4.

Embodiment 37 is the method of embodiment 35 or 36, wherein PAR1, PAR2, PAR3, or PAR4 is endogenously or exogenously expressed.

Embodiment 38 is the method of embodiment 37, wherein PAR1, PAR2, PAR3, or PAR4 is exogenously expressed.

Embodiment 39 is the method of embodiment 38, wherein endogenous PAR1, PAR2, PAR3, or PAR4 expression is substantially eliminated.

Embodiment 40 is the method of any one of embodiments 35-39, wherein the cell is selected from the group consisting of a CHO-K1 cell, a COS-7 cell, and a HEK293 cell.

Embodiment 41 is the method of any one of embodiments 35-40, wherein the agent is selected from the group consisting of a small molecule, a polypeptide, an antibody, a lipid, a polysaccharide, and a polynucleotide.

Embodiment 42 is the method of any one of embodiments 35-41, wherein the control is a cell engineered to express a mutant protease activated receptor polypeptide, preferably wherein the mutant protease activated receptor polypeptide is a mutant PAR2 polypeptide.

Embodiment 43 is the method of embodiment 42, wherein the mutant PAR2 polypeptide comprises an amino acid sequence with at least 95% identity to SEQ ID NO:55.

Embodiment 44 is the method of any one of embodiments 35-43, wherein the agent binds the signal peptide sequence of the PAR1, PAR2, PAR3, or PAR4 intracellularly to disrupt the signal peptide function.

Embodiment 45 is the method of any one of embodiments 35-44, wherein the agent binds an allosteric site on the PAR1, PAR2, PAR3, or PAR4, wherein binding of the agent to the allosteric site disrupts the signal peptide function.

Embodiment 46 is the method of any one of embodiments 35-45, wherein the protease is selected from the group consisting of trypsin, tryptase, factor Xa TF, factor Vila, matriptase/MT-serine protease 1, cysteine proteinase (RgpB), dust mite proteinase Der p3, dust mite proteinase Der p9, furin, and thrombin.

Embodiment 47 is the method of any one of embodiments 35-46, wherein the peptide ligand comprises SLIGKV (SEQ ID NO:1), SLIGRL-NH2 (SEQ ID NO:58), or 2-furoyl-LIGRL-NH2 (SEQ ID NO:59).

Embodiment 48 is the method of any one of embodiments 35-47, wherein the small molecule is GB110.

Embodiment 49 is an isolated mutant PAR2 polypeptide comprising an amino acid sequence selected from the group consisting of SEQ ID NO:45, SEQ ID NO:51, SEQ ID NO:53, and SEQ ID NO:55.

Embodiment 50 is an isolated polynucleotide encoding the mutant PAR2 polypeptide of embodiment 49.

Embodiment 51 is a vector comprising the isolated polynucleotide of embodiment 50.

Embodiment 52 is a host cell comprising the vector of embodiment 51.

Embodiment 53 is a method of producing an isolated mutant PAR2 polypeptide, the method comprising culturing the host cell of embodiment 52 under conditions suitable for the expression of the mutant PAR2 polypeptide and recovering the mutant PAR2 polypeptide from the cell or culture.

### EXAMPLES

Materials and Methods

Reagents

The PAR2 agonist peptide ligand, SLIGKV (SEQ ID NO:1), was synthesized by Innopep, Inc. (San Diego, Calif.). Trypsin (sequencing grade), thrombin, and protease inhibitors were purchased from Sigma Aldrich (St. Louis, Mo.).

Quantitative PCR Analysis of the mRNA Expression Levels of PARs

Total RNAs were isolated from COS7, HEK293, and CHO-K1 cells respectively using an RNA isolation kit (RNeasy Mini Kit) from Qiagen (Hilden, Germany). cDNAs were synthesized from the isolated RNA using a cDNA synthesis kit (Advantage RT-PCR kits) from Clontech (Mountain View, Calif.). Specific primers designed according to human, monkey, and hamster PAR1, PAR2, PAR3, and PAR4 were used to quantify each mRNA expression using a qPCR machine (QuantStudio, ABI) as described (Liu et al., Nature 475:519-23 (2011)). In parallel, primers for β-actin were used to amplify β-actin cDNA as the internal controls. The relative expressions of different PAR mRNAs were normalized using the expression level of β-actin. The qPCR primers were designed based on the published cDNA sequences and the primer sequences are listed in Table 1.

TABLE 1
qPCR primers

| | | SEQ | | SEQ |
|---|---|---|---|---|
| Gene (Accession | | ID | | ID |
| No.) | Forward Primer Sequence | NO: | Reverse Primer Sequence | NO: |
| Human PAR1 | CCATTTTGGGAGGATGAGGA | 2 | AGGATGAACACAACGATGGCC | 3 |
| (NM_001992.4) | G | | AT | |
| Human PAR2 | ATGGCACATCCCACGTCACTG | 4 | GAACCAGATGACAGAGAGGAG | 5 |
| (NM_005242.5) | GA | | GTC | |
| Human PAR3 | ATGCTACCATGGGGTACCTG | 6 | GTTGCCATAGAAGATGACTGTG | 7 |
| (NM_004101.3) | AC | | GT | |
| Human PAR4 | CCTCCACCATGCTGCTGATGA | 8 | AGGTCTGCCGCTGCAGTGTCA | 9 |
| (NM_003950.3) | A | | | |
| Human Actin | GGTCATCACCATTGGCAATG | 10 | GATCTTGATCTTCATTGTGCTG | 11 |
| (NM_001101.4) | AG | | | |
| Monkey PAR1 | CCATTTTGGGAGGATGAGGA | 12 | AGGATGAACACAACGATGGCC | 13 |
| (XM_011730122) | G | | AT | |
| Monkey PAR2 | ATGGCACATCCCACGTCACTG | 14 | GAACCAGATGACAGAGAGGAG | 15 |
| (XM_011730121) | GA | | GTC | |
| Monkey PAR3 | ATGCTACCATGGGGTACCTG | 16 | GTTGCCATAGAAGATGACTGTG | 17 |
| (XM 003899832) | AC | | GT | |
| Monkey PAR4 | CCTCCACCATGCTGCTGATGA | 18 | AGGTCTGCCGCTGCAGTGTCA | 19 |
| (XM_011759280) | A | | | |
| Monkey Actin | GGCACCACACCTTCTACAATG | 20 | GGTCCAGACGCAGGATGGCAT | 21 |
| (NM_001033084) | | | | |
| Hamster PAR1 | CGCCAGCCAGAATCTGAGAT | 22 | CGAGGGGATGAAGAGCCTCAG | 23 |
| (XM_007636187) | G | | | |
| Hamster PAR2 | GGACGCAACGGTAAAGGAAG | 24 | CTTCGTCCGGAAAAGGAAGAC | 25 |
| (XM_007632089) | A | | | |
| Hamster PAR3 | CTTCTGCCAGCCACTTTTTGC | 26 | GGAACTTCTCAGGTATCCCATG | 27 |
| (XM_003498712) | | | GT | |
| Hamster PAR4 | GGGAAATTCTGTGCCAACGA | 28 | GGCCAATAGTAGGTCCGAAAC | 29 |
| (XM_007629105) | C | | | |
| Hamster Actin | GTAGCCATTCAGGCTGTGCTG | 30 | ATGCAGCAGTGGCCATCTCCT | 31 |
| (NM_001244575) | | | | |

Generation of PAR1, PAR2 Knock-Out Cell Line.

A PAR1, PAR2 knock-out HEK293 cell line was created by Applied StemCells (Milpitas, Calif.) using a CRISPR/Cas9 approach. Briefly, the coding region (nucleotide 374-643) of PAR1, which encodes the protein region transmembrane region 2 (TM2) to transmembrane region 3 (TM3) of PAR1, was deleted. Similarly, the coding region (281-627) of PAR2, which encodes the protein region TM2 to TM3 of PAR2, was deleted. Single cell clones were isolated. PCR analysis of the genomic DNA followed by DNA sequencing was used to confirm the deletion of the DNA fragments.

Molecular Cloning of PAR2 Constructs.

The PAR2 coding region was amplified by polymerase chain reaction (PCR) using primers (5′ atg tct GAA TTC GCC ACC atg cgg agc ccc agc gcg gcg tgg ctg ctg-3′ (SEQ ID NO:32); reverse primer: 5′-atg tct GCG GCC GCt caa tag gag gtc tta aca gtg gtt gaa ct-3′ (SEQ ID NO:33)) designed based on the published PAR2 coding sequence (Genbank Accession No. NM 005242.5). Human colon cDNA purchased from Clontech (Palo Alto, Calif.) was used as the template. Expanded high fidelity PCR system (Roche Life Science, Indianapolis, Ind.) was used to amplify the full length PAR2 cDNA coding region. The resulting DNA was digested using EcoR1 and Not1 restriction enzymes (Promega, Madison, Wis.) and then cloned into pcDNA3.1 (Invitrogen, Carlsbad, Calif.). The insert region was then sequenced by Eton Biosciences (San Diego, Calif.) and the identity of the entire coding region was confirmed.

Expression constructs for PAR2 with an Arg36Ala mutation (PAR2(R36A)) (SEQ ID NO:55), PAR2 without the signal peptide (PAR2ΔSP) (SEQ ID NO:45), PAR2ΔSP with an Arg36Ala mutation (PAR2ΔSP(R36A)) (SEQID NO:53), and PAR2 without the signal peptide and the tethered ligand (PAR2ΔSPΔL) (SEQ ID NO:51) were generated by site directed mutagenesis using overlapping PCR approach (Maher et al., Pharmacol. Exp. Ther. 357:394-414 (2016))

Genes for PAR2 with the signal peptide coding regions replaced by the insulin signal peptide, or the insulin receptor signal peptide were synthesized by Eton Biosciences (San Diego, Calif.). Similarly, expression constructs for various PAR2 variants with a GFP fused to the C-termini, human IgG-Fc coding region with or without a PAR2 signal peptide coding region, with an insulin, or with an insulin receptor signal peptide coding region were synthesized. The genes were cloned into pcDNA3.1 and the entire coding regions were sequenced to confirm the identities.

Intracellular Ca²⁺ Mobilization Assay

FLIPR-Tetra (Molecular Device, San Jose, Calif.) was used to monitor intracellular Ca²⁺ mobilization in HEK293 cells, HEK293 cells with PAR1 and PAR2 knocked-out, and cells transiently transfected with various PAR2 expression constructs. Cells were grown in 96-well polyD-lysine coated black FLIPR plates (Corning) in DMEM supplemented with 10% FCS, 1 mM pyruvate, 20 mM HEPES, at 37° C. with 5% CO₂. For transient transfection, cells were grown in 96-well polyD-lysine coated black FLIPR plates and transfected using FuGENE HD (Promega, Madison, Wis.) as the transfection reagent according to the manufacturer's instructions. For samples treated with protease inhibitors, protease cocktail was added to cell culture one day after transfection and incubated overnight. Two days after transfection, cell culture media were removed, and cells were washed using HMS buffer plus 20 mM HEPES. Ca²⁺ dye (Flura 3) diluted in HMS buffer plus 20 mM HEPES was used to incubate cells at RT for 40 minutes to allow Ca²⁺ to enter cells. Intracellular Ca²⁺ mobilization stimulated by various concentrations of ligands (trypsin, or peptide ligand) was monitored by FLIPR-Tetra as described (Liu et al., Mol. Pharmacol. 88:911-25 (2015)). The untransfected cells were used as negative controls.

Enzyme Linked Immunosorbent Assay (ELISA) for the Measurement of IgG-FC Secretion

COST cells were grown in 6 well plates with DMEM supplemented with 10% FCS, 1 mM pyruvate, 20 mM HEPES, at 37° C. with 5% CO₂ and transfected by different expression constructs for human IgG-Fc with various signal peptide coding regions using LipofectAmine (Invitrogen, Carlsbad, Calif.) as the transfection reagent according to the manufacturer's instructions. Untransfected cells were used as negative controls. To measure the secreted human IgG-FC in the medium, one day after transfection, the cells were washed 3 times using PBS and then cultured in serum free DMEM plus 1 mM pyruvate and 20 mM HEPES. Three days after transfection, the conditioned media from the transfected cells were harvested and centrifuged at 10,000 g at 4° C. for 20 minutes to remove the cell debris. 50 μl of the conditioned medium from each transfection was incubated in one well of a 96-well ELISA plate (UltraCruz® ELISA Plate, high binding, 96 well, Flat bottom, Santa Cruz Biotechnology; Dallas, Tex.) at 37° C. for 1 hour to allow protein in the media to adsorb to the plates. The plates were washed 3 times using PBS+0.1% Tween-20 (PBST), blocked using 3% no-fat milk in PBST for 30 minutes at RT, and then incubated using HRP-conjugated goat-anti-human Ig-GF antibody (50 ng/ml) diluted in 3% no-fat milk in PBST at 4° C. overnight. The plates were washed 3 times using PBST and then developed using an ELISA developing kit (BD Biosciences; San Jose, Calif.). The optical densities at 450 nm were read using an ELISA plate reader (Molecular Devices; San Jose, Calif.).

To measure intracellular IgG-Fc protein, one day after transfection, cells were trypsinized and seeded in 96-well culture plates (30,000 cell/well) and grown in DMEM supplemented with 10% FCS, 1 mM pyruvate, 20 mM HEPES. Three days after transfection, the media were removed, and cells were washed using PBS, and then fixed by 10% formaldehyde in PBS at RT for 15 minutes. The cells were penetrated using 1% Triton-X-100 at RT for 10 minutes and blocked using 3% no-fat milk in PBST for 30 minutes at RT. The cells were then incubated using HRP-conjugated goat-anti-human IgG-Fc antibody, and the plate was developed and read as described above.

Immuno Fluorescent Staining of Intracellular IgG-Fc

COS7 cells were transfected with various IgG-Fc expression constructs. One day after transfection, cells were trypsinized and seeded in a 4-well cell culture chamber slides (Stellar Scientific, Baltimore, Md.) (60,000 cells/well). Three days after transfection, the media were removed, and cells were washed using PBS, and then fixed by 10% formaldehyde in PBS at RT for 15 minutes. The cells were penetrated using 1% Triton-X-100 at RT for 10 minutes and blocked using 3% no-fat milk in PBST for 30 minutes at RT. The cells were then incubated using FITC-labelled goat-anti-human IgG-FC antibody (ThermoFisher Scientific; Waltham, Mass.) (200 ng/ul) diluted in 3% no-fat milk in PBST at 4° C. overnight. The slides were then washed 3 times using PBST, dried using cool air, and viewed under a fluorescent microscope.

Identification of the Signal Peptide Cleavage Site of PAR2

COS7 cells were grown in 15 cm dishes in DMEM supplemented with 10% FCS, 1 mM pyruvate, 20 mM HEPES, at 37° C. with 5% CO₂. The cells were transfected with the expression construct of human IgG-FC with the N-terminus of PAR2 using LipofecAmine. One day after transfection, the cells were washed 3 times using PBS and then cultured in serum free Opti-MEM (Life Technology) plus Pen/Strep. Three days after transfection, the media were collected and centrifuged to remove the cell debris. The supernatants were passed through a Protein A (Sigma) affinity column. The column was washed with PBS, eluted using 0.1 M Glycine/HCl (pH 2.8), and then neutralized using 1 mM Tris-HCl, pH 8.0. The eluted protein was first treated with PNGase-F (Promega) to remove the N-linked glycosylation and then analyzed by mass spectrometry to determine the N-terminal sequence. Protein sequencing was performed using a generic in-solution protein digestion and LC-MS/MS method. Briefly, a 10 μl protein sample in 50 mM ammonia bicarbonate buffer (pH 7.8) was reduced by 11.3 mM dithiothreitol at 60° C. for 30 minutes (without urea), alkylated with 37.4 mM iodoacetamide (RT, 45 minutes), and then digested with 0.2 μg Trypsin (37° C., overnight). LC/MS analysis was carried out on an Agilent 1290 UHPLC coupled to a 6550 qTOF mass spectrometer, under the control of MassHunter software version 4.0. Chromatography was run with an Agilent AdvanceBio Peptide Map column (2.1×100 mm, 2.7 μm) using water/acetonitrile/0.1% formic acid as mobile phases, and mass spectrometric data were acquired in both MS and MSMS modes.

Protease Inhibitor Treatment of Cells Recombinantly Expressing PAR2 Receptors

The wild type and various mutant PAR2 variant expression constructs were transiently transfected into HEK293 cells with par1 and part genes knocked-out. 24 hours after transfection, cells were treated for 12 hours with a protease inhibitor cocktail including 4-(2-aminoethyl)benzenesulfonyl fluoride hydrochloride (AEBSF, 500 uM), Leupeptin (50 uM), aprotinin (50 uM).

Measurement of the Total and Cell Surface Expression of PAR2 by ELISA

ELISA was used to measure the total and cell surface PAR2 protein expression. The wild type and different mutant PAR2 variants were transiently expressed in HEK293 cells with the endogenous PAR1 and PAR2 knocked-out. The cells were transfected in 10 cm cell culture dishes and, 24 hours after transfection, split into a 96-well polyD-lysine coated plate. 48 hours post transfection, cells were fixed as described above. To measure the total PAR2 expression, the fixed cells were penetrated using 1% triton-X-100, blocked with 3% no-fat milk, and then incubated with a monoclonal antibody (3 μg/ml, mouse anti-human PAR2 (BioLegand, San Diego, Calif.)), which recognizes the N-terminal region (amino acid residues 37-62) of the human PAR2, at 4° C. overnight. The plate was washed with cold PBS 3 times and then incubated using a HRP-conjugated goat-anti-mouse IgG secondary antibody (30 ng/ml, Pierce) at RT for 1 hour. The plate was washed again using PBS and developed using an ELISA developing kit as described above. To measure the cell surface PAR2 expression, the ELISA assays were performed in the same manner as the total PAR2 measurement without using triton-X-100 as the cell penetrating agent.

Measurement of the Total Expression and Cellular Localization of PAR2-GFP Fusion Proteins

GFP fusion proteins of PAR2 wild type and various mutants were transiently expressed in 96-well poly-D-lysine plates in HEK293 cells with the endogenous PAR1 and PAR2 knocked-out as described above in methods for Intracellular Ca²⁺ mobilization assay. 48 hours after transfection, the media were aspirated, and cells were fixed using 4% Paraformaldehyde in PBS (Sigma; St. Louis, Mo.). The fluorescent intensities of the cells were read using an Envision plate reader (PerkinElmer; Waltham, Mass.). The fixed cells were then analyzed using a confocal microscopy for PAR2 cellular localizations.

Results and Discussion

PAR2 Signal Peptide Behaves as a Classical Signal Peptide

PAR2 Signal Peptide Leads IgG-Fc Fragment Secretion to the Medium.

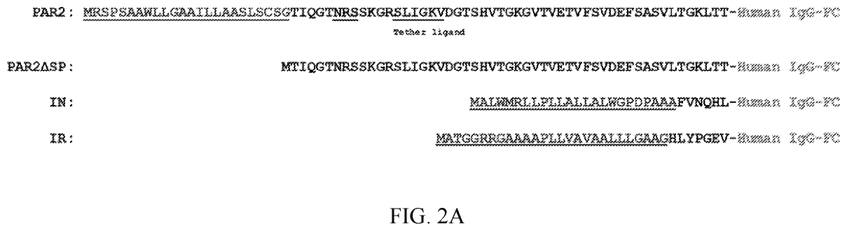

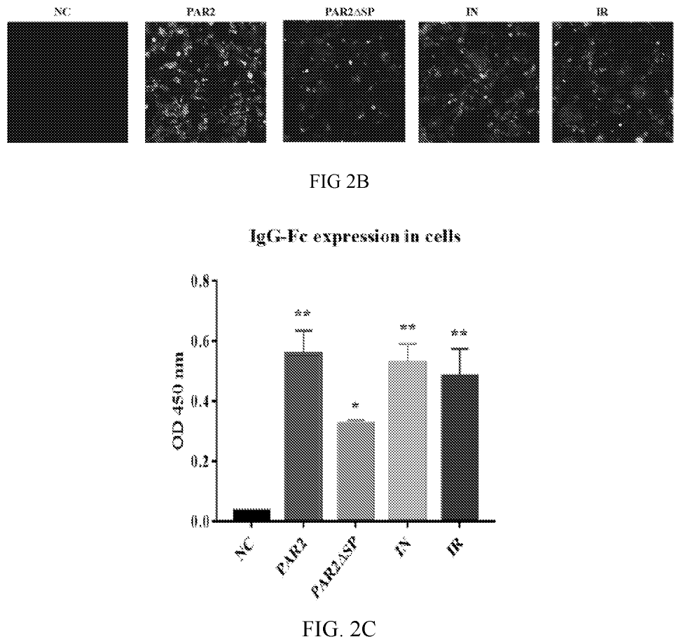

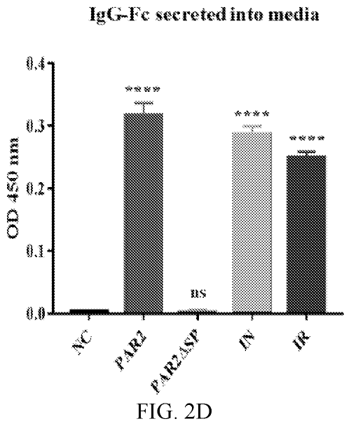

A classical signal peptide is typically found at the N-termini of either secreted proteins (such as insulin) or cell surface proteins (such as insulin receptor). It typically consists of a stretch of 20-30 hydrophobic amino acid residues. Its known function is to help a secreted or a cell surface protein to target the ER during protein translation and cross the plasma membrane. PAR2 has a predicted signal peptide sequence at its N-terminus, and it was hypothesized to function as a classical signal peptide. To address this, a few expression constructs were devised (FIG. 2A) to test whether the signal peptide of PAR2 enables the secretion into the cell culture medium of human IgG-Fc fragment, which lacks a signal peptide. IgG-Fc was used as a control due to the ease of detection with an ELISA assay or immune-staining. When recombinantly expressed in mammalian cells, without a signal peptide, IgG-Fc is only expressed intracellularly. In contrast, with a signal peptide, IgG-Fc can be secreted into the cell culture medium. One IgG-Fc construct contained the N-terminus of PAR2 with its signal peptide (SEQ ID NO:34 (DNA); SEQ ID NO:35 (protein)), and another IgG-Fc construct contained the PAR2 N-terminus in which its signal peptide was deleted (SEQ ID NO:36 (DNA); SEQ ID NO:37 (protein)). Constructs with an insulin signal peptide (a secreted protein signal peptide) or an insulin receptor signal peptide (a cell surface receptor signal peptide) fused to human IgG-Fc (SEQ ID NO:38 (DNA); SEQ ID NO:39 (protein) and SEQ ID NO:40 (DNA); SEQ ID NO:41 (protein), respectively) were also used as positive controls in the experiment. Immuno-staining (FIG. 2B) and ELISA (FIG. 2C) were used to detect and measure IgG-Fc expression in the transfected cells and demonstrated that all cells transfected with various IgG-Fc expression constructs expressed IgG-Fc in the cells. It was demonstrated that fusing the N-terminus of PAR2 with the PAR2 signal peptide to the human IgG-Fc, effectively led to the secretion of IgG-Fc to the medium, thus functioning similarly to that of the insulin signal peptide or the insulin receptor signal peptide (FIG. 2D). In contrast, fusing the PAR2 N-terminus without the PAR2 signal peptide failed to lead to the secretion of IgG-Fc into the medium.

PAR2 Signal Peptide is Cleaved from the Mature Protein

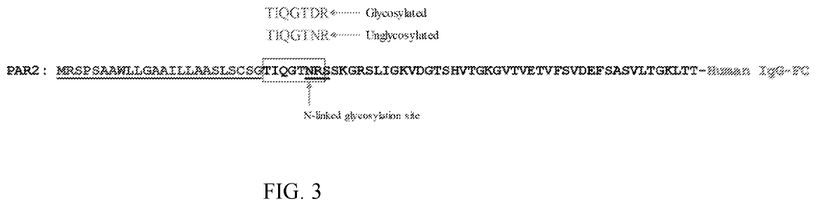

It has been reported that for CRF2(a) receptor, the signal peptide may not be cleaved from the mature proteins following membrane insertion (Teichmann et al., J B C 287:27265-74 (2012)). To determine if this was the case for the PAR2 signal peptide, it was examined whether the signal peptide of PAR2 was cleaved from the mature IgG-Fc protein with the PAR2 N-terminus following secretion. The conditioned medium from the COS7 cells transfected with the expression construct for PAR2 N-terminus fused to IgG-Fc was collected (FIG. 2A). Secreted PAR2-IgG-Fc fusion protein was affinity purified, glycosylation moieties were removed, and then analyzed by mass spectrometric (MS) protein sequencing. The results demonstrated that the most N-terminal sequence that matches PAR2 sequence is TIQGTNR (SEQ ID NO:42) (FIG. 3), suggesting that the signal peptide had been cleaved following the protein secretion, with the cleavage site being between residues Gly24 and Thr25. Interestingly, a variant sequence TIQGTDR (SEQ ID NO:43) was also observed. This sequence differs from TIQGTNR by one residue (from N to D). Since the residue Asn30 is a part of a NRS sequence (a N-linked glycosylation site, FIG. 3) and glycosylated Asn residues are converted to Asp after de-glycosylation by PNGase-F, the results suggested that at least part of the expressed protein is glycosylated at this N-linked glycosylation site.

PAR2 Signal Peptide is Important for PAR2 Receptor Functional Expression and Activation by its Ligands.

Generation of a PAR1 and PAR2 Knock-Out HEK293 Cell Line for Recombinant Expression and Characterization of PAR2 Receptor.

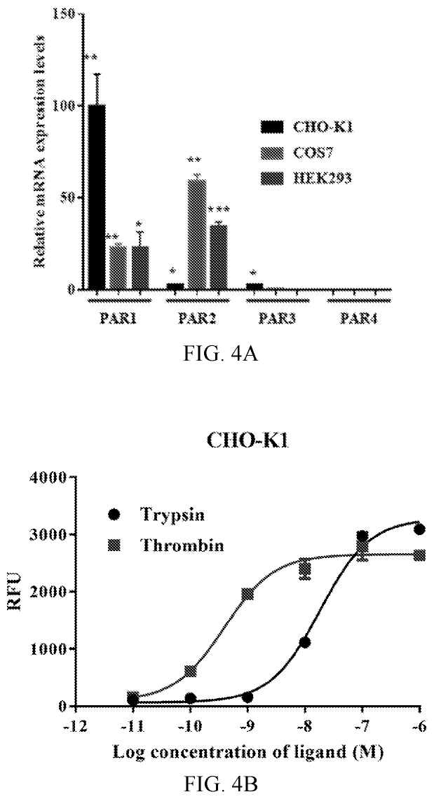

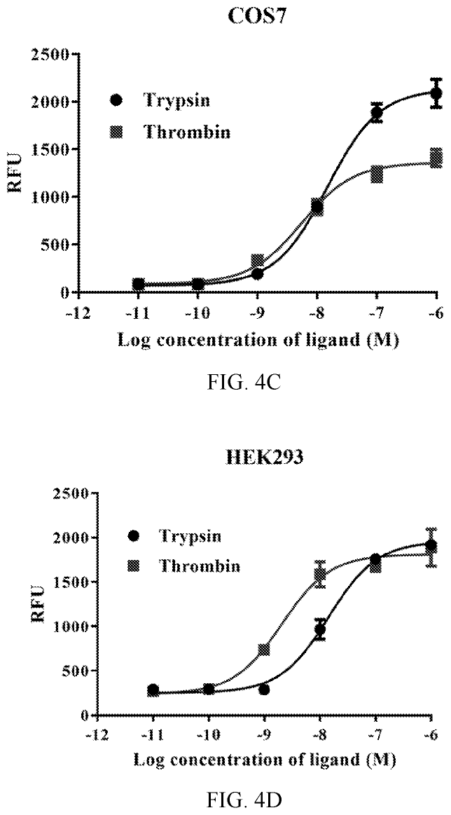

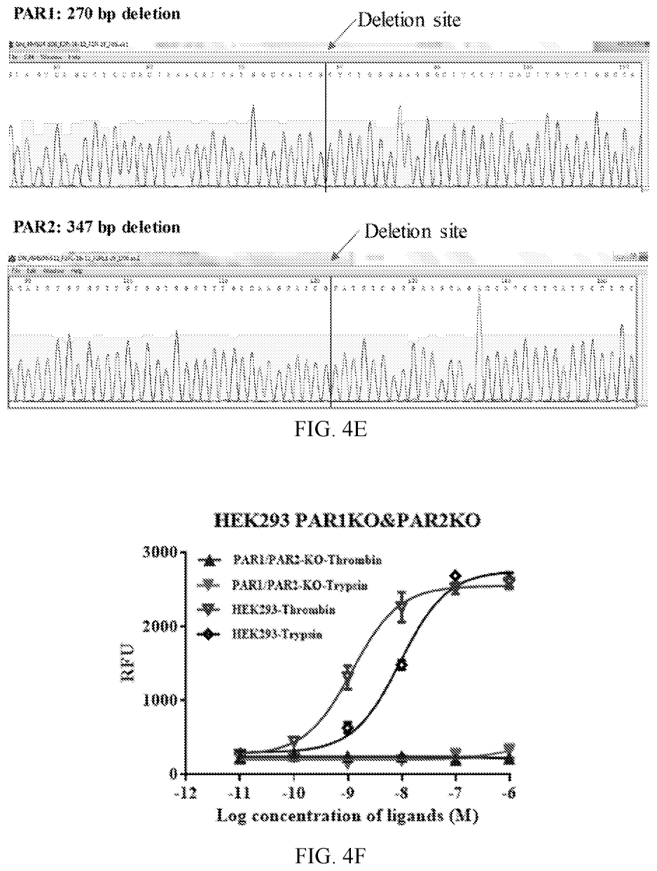

To evaluate receptor localization and function of recombinant PAR2, it was essential to have a host mammalian cell line that did not express endogenous PAR2 or other PAR receptors. Mammalian cells were tested for recombinant expression, including HEK293, CHO-K1, and COS7 cells, and it was found that all three cell lines express relatively high PAR1 and PAR2 mRNA (FIG. 4A). In addition, in functional assays, the cell lines all responded to PAR1 and PAR2 ligands (thrombin and trypsin, respectively) (FIGS. 4B-4D). Since the presence of naturally expressed PAR1 and PAR2 in these host cells could complicate the characterization of the recombinantly expressed PAR2, a HEK293 cell line, which does not express PAR3 and PAR4, with both par1 and par2 genes knocked-out by CRISPR/cas9 was created (FIG. 4E). Pharmacological characterization of this cell line demonstrated that the loss of both par1 and par2 led to a lack of response to the PAR1 ligand, thrombin, or the PAR2 ligand, trypsin (FIG. 4F). These cells were then used to study expression and localization of recombinant PAR2.

Deletion of the Signal Peptide Reduced the Functional Expression of PAR2, which can be Rescued by a Replacement Signal Peptide.

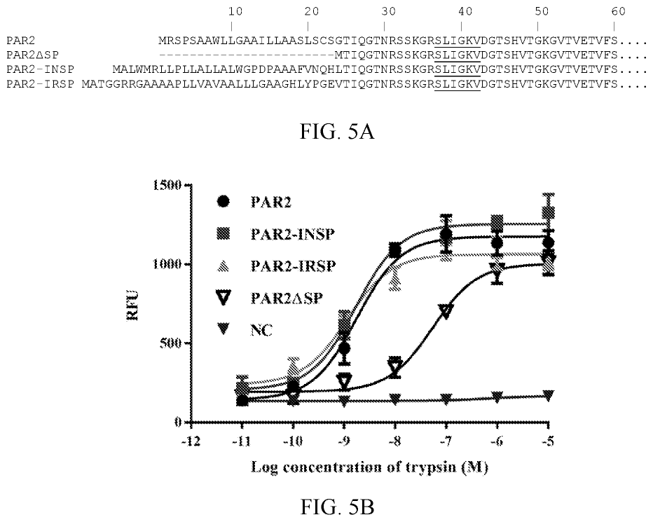

To assess the functional role of the PAR2 signal peptide, several modifications were made to the PAR2 N-terminus, including a N-terminal deletion to remove the signal peptide (PAR2ΔSP) (SEQ ID NO:45) and the replacement of the PAR2 signal peptide with an insulin signal peptide (PAR2-INSP) (SEQ ID NO:47), or an insulin receptor signal peptide (PAR2-IRSP) (SEQ ID NO:49) (FIG. 5A). Pharmacological characterization of the modified receptors using FLIPR assay showed that the recombinantly expressed PAR2 responds to trypsin (EC₅₀=1.5 nM) and PAR2 agonist peptide (PAR2-AP) (EC₅₀=50 nM) with much higher sensitivity compared to the endogenously expressed PAR2 in HEK293 cells (EC₅₀=10 nM for trypsin and EC₅₀=1.5 uM for PAR2-AP) (FIGS. 5B and 5C). This is due to the over expression of the recombinant receptor causing a super-pharmacology phenomenon (Kenakin, Trends Pharmacol. Sci. 18:456-64 (1997)). In this case, the EC₅₀ value was a good indicator of the relative number of receptors at the cell surface. Compared to the cells expressing the wild type PAR2, cells expressing PAR2 without its signal peptide demonstrated dramatically reduced sensitivity to both trypsin and PAR2-AP (EC₅₀ for trypsin: 50 nM; EC₅₀ for PAR2-AP: 5.8 μM), suggesting the signal peptide is an important component of PAR2 functional cell surface expression. Supporting this hypothesis, replacement of the PAR2 signal peptide either with a signal peptide from insulin or from the insulin receptor fully restored the receptor ligand sensitivity (FIG. 5).

Tethered Ligand Necessitates PAR2 Signal Peptide.

Further Deletion of the Tethered Ligand Region Rescues the Functional Expression of PAR2 without the Signal Peptide.

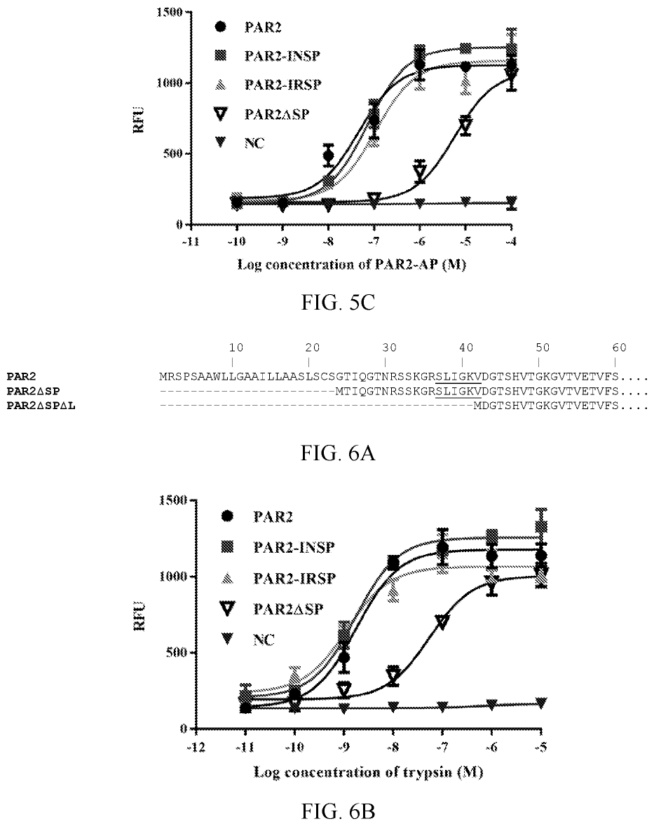

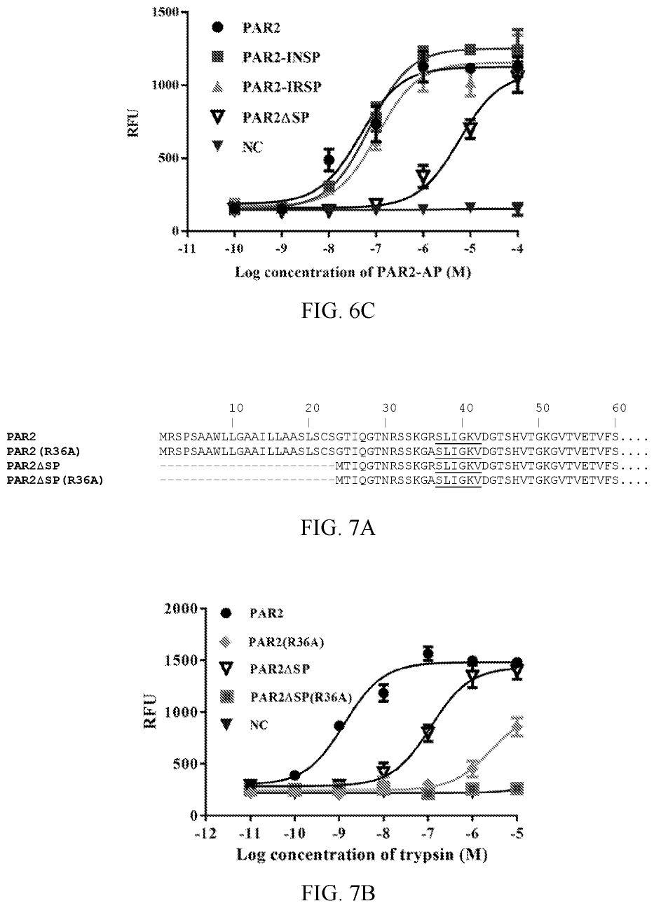

PARS are activated by proteases, which generate new N-termini and expose the tethered peptide ligands present in the N-terminal extracellular regions of the receptors. This unique receptor activation mechanism, combined with the fact that signal peptide-less PAR2 had a poor response to ligand stimulation, led to speculation that the necessity of the signal peptide for PAR2 could be related to the presence of the tethered ligand. A signal peptide-less PAR2 mutant with a further deletion to the region of the tethered ligand (PAR2ΔSPΔL) (SEQ ID NO:51) was constructed (FIG. 6A). This mutant receptor lacks the signal peptide and the tethered ligand sequence (SLIGKV) (SEQ ID NO:1) and was not activated by trypsin, however it could be fully activated by the synthetic agonist peptide PAR2-AP (SEQ ID NO:1) similarly to the wild type PAR2 receptor in the FLIPR assay (FIG. 6B). This suggests that further deletion of the tethered ligand sequence (SLIGKV) restored functional cell surface expression of PAR2 without the signal peptide. The results also suggest that, without a signal peptide, PAR2 could be susceptible to unintended intracellular protease activation, leading to poor functional cell surface expression.

Mutation of Arg36 to Ala, which Blocks the Trypsin Activation Site, Increased the Functional Expression of PAR2 without the Signal Peptide.

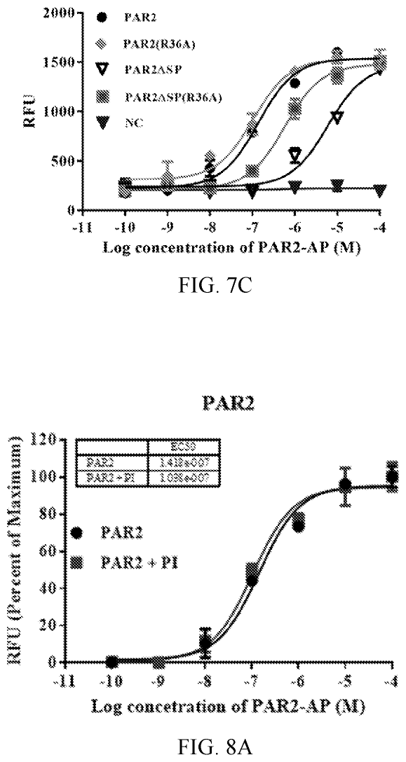

Trypsin activates PAR2 by cleaving after residue Arg36. This generates a new N-terminus (with sequence SLIGKV---), which serves as a tethered ligand to activate the receptor. Mutating Arg36 to Ala prevents the trypsin cleavage at this position, and therefore blocks trypsin-mediated receptor activation. A mutation at the Arg36 position on PAR2 without the signal peptide (PAR2ΔSP(R36A)) (SEQ ID NO:53) was made, and this construct was tested to determine if this mutation changed the level of functional receptor expression. In parallel, the same mutation on the full length PAR2 receptor (PAR2(R36A)) (SEQ ID NO:55) was made, and these receptors were characterized in FLIPR assays after stimulation with trypsin and PAR2-AP. The results demonstrated that the Arg36Ala mutation blocked, as expected, trypsin activation of PAR2 without the signal peptide (FIG. 7A). However, when the PAR2-AP was used as the ligand, the mutant receptor (PAR2ΔSP(R36A)) (SEQ ID NO:55) demonstrated a much higher sensitivity to PAR2-AP compared to that of PAR2ΔSP (SEQ ID NO:45) (FIG. 7B). As a control, the same mutation in full length PAR2 receptor (PAR2(R36A)) (SEQ ID NO:55), which responded to trypsin stimulation very poorly (FIG. 7B), responded to PAR2-AP stimulation almost identically to the full length PAR2 receptor (FIG. 7C). The small but detectable activation of PAR2(R36A) (SEQ ID NO:55) by trypsin (FIG. 7B) could be due to the cleavage of PAR2 by trypsin at Arg³¹, or Lys³⁴ positions, resulting in tethered ligands with poor activity for receptor activation.

Protease Inhibitor Treatment Increased Functional Expression of PAR2 without the Signal Peptide.

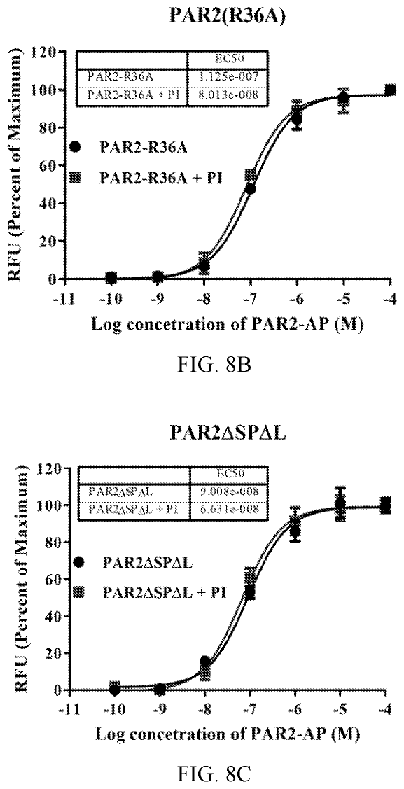

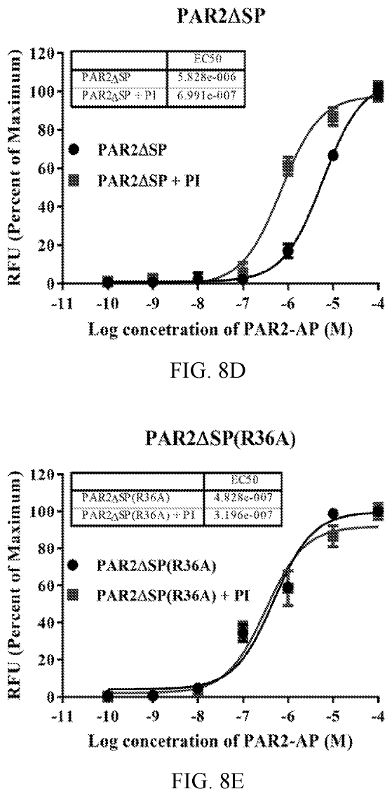

Serine protease inhibitors were hypothesized to help the functional expression of PAR2 without a signal peptide by blocking premature intracellular protease-mediated activation. A protease cocktail including AEBSF, Leupeptin, and aprotinin was used to inhibit ER and Golgi proteases (Okada, et al., J. Biol. Chem. 278:31024-32 (2003); Wise et al., Proc. Natl. Acad. Sci. USA 87:9378-82 (1990)). Cells expressing the wild type PAR2 and various mutant forms of PAR2 were treated with the protease inhibitor cocktail and then tested for their responses to PAR2-AP stimulations. Trypsin was not used in this assay because trypsin is inhibited by the protease inhibitor cocktails. The results demonstrated that while protease inhibitors did not affect the EC₅₀ values of PAR2-AP stimulated responses for PAR2 wild type (SEQ ID NO:57), PAR2(R36A) (SEQ ID NO:55), PAR2ΔSP(R36A) (SEQ ID NO:53), and PAR2ΔSPΔL (SEQ ID NO:51), the protease cocktail clearly increased functional expression of PAR2ΔSP (SEQ ID NO:45) by decreasing the EC₅₀ value (from 5.8 μM to 0.7 μM) (FIG. 8).

Arg36Ala Mutation and the Protease Inhibitor Treatment Increase the Cell Surface Expression of Signal Peptide-Less PAR2.

To confirm whether the reduced responses of signal peptide-less PAR2 to the ligand stimulation is due to a lack of total receptor protein expression, and/or a lack of cell surface expression, a monoclonal antibody against amino acid residues 37-62 of PAR2 was used in ELISA assays to measure the total and cell surface expression of the various forms of PAR2, and to determine the effect of protease inhibitor treatment. It was observed that PAR2 wild type (SEQ ID NO:57) and PAR2(R36A) (SEQ ID NO:55) mutants had the highest total and cell surface protein expression as measured by ELISA. PAR2ΔSPΔL (SEQ ID NO:51) had slightly lower expression compared to that of the PAR2 wild type (SEQ ID NO:57) in both total and cell surface expression. As this variant of PAR2 is missing amino acid residues 1-42, the reduced detection of protein expression could be due to the poor antibody recognition. PAR2ΔSP(R36A) (SEQ ID NO:53) had lower total and cell surface expression, and PAR2ΔSP (SEQ ID NO:45) had the lowest total and cell surface expression levels (FIG. 9). The data showed that the great majority of PAR2ΔSP (SEQ ID NO:45) protein was located intracellularly and only a small portion of it was present on the cell surface. For PAR2 wild type (SEQ ID NO:57), PAR2(R36A) (SEQ ID NO:55), and PAR2ΔSPΔL (SEQ ID NO:51), over 90% of the proteins were present on the cell surface. Corroborating the functional assays, protease inhibitor treatments increased the total level, and especially the cell surface expression levels for PAR2ΔSP (SEQ ID NO:45) while having little or no effect on the protein expression of other forms of PAR2 proteins (FIG. 9). Stimulation of receptors using PAR2 peptide ligand (PAR2-AP) (SEQ ID NO:1) decreased the cell surface and the total protein expression levels for all variants of PAR2 except PAR2ΔSP (SEQ ID NO:45). This was likely due to that the majority of PAR2ΔSP (SEQ ID NO:45) being intracellular, and the ligand stimulation of the cell surface receptor, causing the subsequent internalization and degradation of the stimulated receptors, applied less to PAR2ΔSP (SEQ ID NO:45).

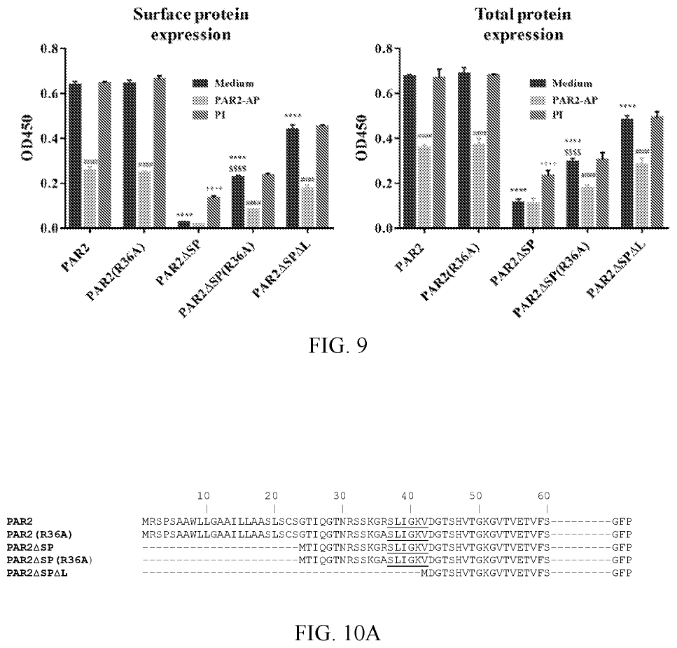

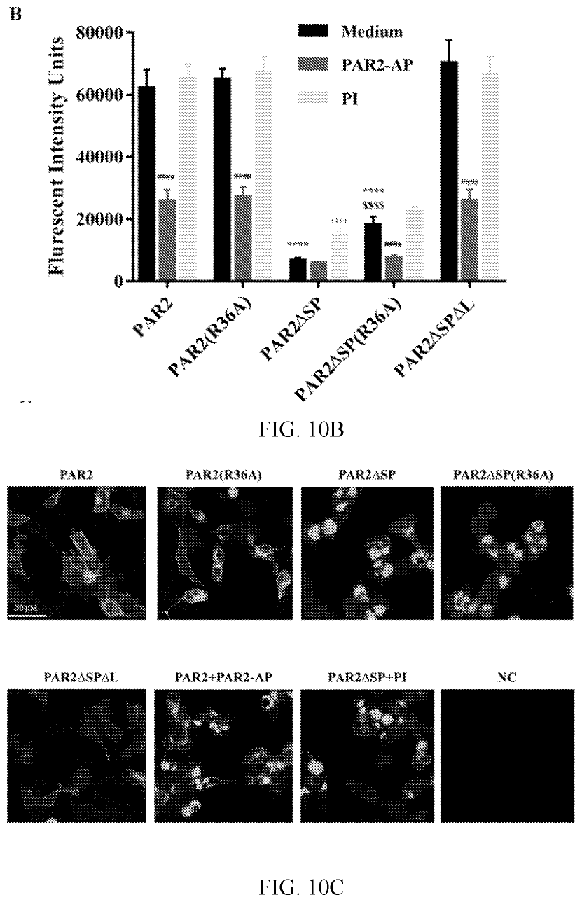

In parallel, to further facilitate the measurements of the protein expression and visualization of protein cellular localizations, various PAR2 expression vectors were constructed by fusing a GFP tag to the C-termini of the PAR2 wild type protein and the various PAR2 mutants (FIG. 10A). The PAR2 expression vectors were subsequently expressed in the par1 and part null HEK293 cell line. The total expression levels of PAR2 and the mutant proteins were measured by measuring GFP fluorescence intensity of the various GFP fusion proteins. In general, the results were similar to that shown by ELISA using the anti-PAR2 antibody except for PAR2ΔSPΔL (SEQ ID NO:51), which showed lower levels compared to that of PAR2 wild type (SEQ ID NO:57) in the ELISA assays, but showed similar expression levels to that of PAR2 wild type (SEQ ID NO:57) in GFP intensities (FIG. 10B). This result supported the earlier speculation that the reduced detection of PAR2ΔSPΔL (SEQ ID NO:51) expression is likely due to the poor recognition of PAR2ΔSPΔL (which missed amino acid residues 37-42 of the recognition site) by the antibody.

To investigate the cellular localizations of PAR2 protein and its variants, confocal microscopy was utilized to analyze the cells that express various PAR2 proteins at various conditions including the treatments with PAR2 ligand or protease inhibitors. PAR2 wild type (SEQ ID NO:57), PAR2(R36A) (SEQ ID NO:55), and PAR2ΔSPΔL (SEQ ID NO:51) proteins were localized on the plasma membranes (FIG. 10C). PAR2ΔSP (SEQ ID NO:45) was only found intracellularly with little to none located on the plasma membranes, which was similar to PAR2 wild type (SEQ ID NO:57) receptor stimulated by the peptide ligand (PAR2+PAR2-AP, FIG. 10C). For PAR2ΔSP(R36A) (SEQ ID NO:53), a portion of protein was expressed on the plasma membrane and a significant amount of protein was also found intracellularly. Interestingly, protease inhibitor treatment enabled the plasma membrane expression of PAR2ΔSP (SEQ ID NO:45) (PAR2ΔSP+PI, FIG. 10C).

Overall, the observed GFP-tagged protein cellular distribution was in agreement with the ELISA data (FIG. 9). Interestingly, the cells expressing PAR2ΔSP(R36A) (SEQ ID NO:53) and protease inhibitor-treated cells expressing PAR2ΔSP (SEQ ID NO:45) appeared to belong to two subcategories. One population of cells had good PAR2 plasma membrane localization, mimicking the wild type PAR2, and another population of cells only had intracellular PAR2, which was similar to that of PAR2ΔSP (SEQ ID NO:45) without the protease inhibitor treatment. The Arg36Ala mutation and the protease inhibitor cocktails (used in the assays) blocked PAR2 cleavage/activation by the serine proteases. However, cells can express other proteases that can cleave and activate PAR2ΔSP intracellularly but would not be blocked by the mutation or the protease inhibitor treatment. It is possible that cells express different proteases under different conditions such as different cell cycle stages (McGrath et al., 2006; Kelly et al., 1998; Goulet et al., 2004; Taylor et al., 2002; Di Bacco et al., 2006; Ly et al., 2014; Yamanaka et al., 2000; Petersen et al., 2000).

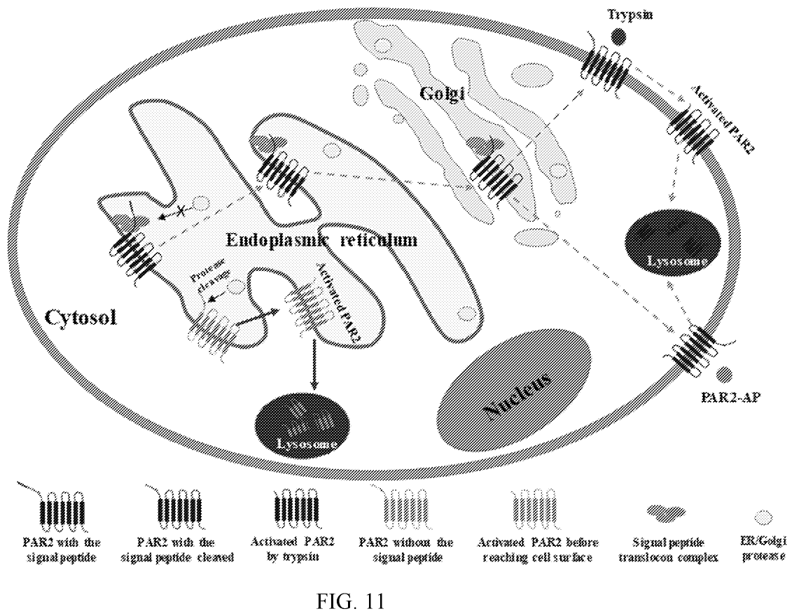

GPCRs are synthesized in the endoplasmic reticulum (ER) and transported to Golgi apparatus and then to the plasma membrane. There are many proteases present in the endoplasmic reticulum and Golgi apparatus (Okada et al., J. Biol. Chem. 278:31024-32 (2003); Otsu et al., J. Biol. Chem. 270:14958-61 (1995); Szabo and Bugge, Annu. Rev. Cell. Dev. Biol. 27:213-35 (2011); Gregory et al., PLoS One 9:387675 (2014); Loo et al., J. Biol. Chem. 273:32373-6 (1998)) which may cleave the protease-sensitive PAR2 activation site at Arg36 position during the protein synthesis and transportation process. This would cause unintended or premature receptor activation, which would subsequently lead to receptor internalization and degradation. The signal peptide of PAR2 is important for its functional expression. However, the removal of the tethered ligand or the blockage of the receptor activation by proteases dismissed the need for the signal peptide, suggesting that the signal peptide may help prevent this unintended cleavage of PAR2 at the activation site during the protein synthesis and/or transportation process. For cell surface proteins using signal peptides, their translocation to ER and eventually the plasma membrane is mediated by ER translocons (Johnson, et al, Cell Dev. Biol. 15:799-842 (1999); Nikonov et al., Biochem. Soc. Trans. 31:1253-6 (2003)), which play roles in protein compartmentalization (Scheele et al., J. Cell. Biol. 87:611-28 (1980); Levine et al., Mol. Biol. Cell 16:279-91 (2005); Schnell et al., Cell 112:491-505 (2003); Shaffer et al., Dev. Cell 9:545-54 (2005); Katerina et al., Mol. Biol. Cell 14:4427-36 (2003)) and segregation (Nikonov et al., Biochem. Soc. Trans. 31:1253-6 (2003); Lu et al., Proc. Natl. Acad. Sci. USA 115:9557-62 (2018); Möller et al., Proc. Natl. Acad. Sci. USA 95:13425-430 (1998)). Although the mechanism remains unclear, ER translocons may play the role in protecting PAR2 from protease cleavage (FIG. 11).

The classical signal peptide has been known to help secreted proteins and cell surface proteins to cross or become embedded in the cell membranes. As indicated above, through studying the signal peptide of PAR2, a function of the signal peptide was observed to serve as a protector of PAR2 from intracellular protease activation. Cleavage of PAR2 by intracellular proteases can lead to the unintended activation of the receptor and the loss of function to sense the extracellular signals. Therefore, with the protease-protection function, the signal peptide can be critical for the function of the PAR2 receptor.

To summarize, the deletion of the signal peptide of PAR2 was observed to decrease PAR2 cell surface expression with the most receptors accumulating intracellularly. However, further deletion of the tether ligand of PAR2, which disabled the activation of PAR2 by trypsin, restored the receptor cell surface expression, suggesting that the necessity of the signal peptide for PAR2 is related to the presence of the tether ligand sequence and the protease activation mechanism. It is hypothesized that the signal peptide of PAR2 protects PAR2 from intracellular protease cleavage and activation. Without the signal peptide, PAR2 can be cleaved and activated by intracellular proteases in the endoplasmic reticulum or Golgi apparatus, leading to the unintended, premature receptor activation and resulting in intracellular accumulation. Supporting this hypothesis, an Arg36Ala mutation at the trypsin activation site, as well as protease inhibitor treatments, both increased the cell surface expression of the signal peptide-less PAR2 and functional responses to ligand stimulation. These results extended the knowledge of PAR2 expression/function and revealed a new role of the signal peptide in protecting cell surface proteins, and perhaps the secreted proteins as well, from intracellular protease cleavages.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of identifying an agent that activates a protease-activated receptor 2 (PAR2) intracellularly, the method comprising:
a. providing a cell expressing the PAR2 on a surface of the cell, wherein the PAR2 comprises a signal peptide sequence;
b. contacting the cell with an agent;
c. measuring a level of PAR2 on the surface of the cell, wherein a reduction in the level of PAR2 on the surface of the cell as compared to a control indicates that the agent is capable of activating PAR2 intracellularly.

2. The method of claim 1, wherein PAR2 is endogenously expressed.

3. The method of claim 1, wherein PAR2 is exogenously expressed.

4. The method of claim 3, wherein endogenous PAR2 expression is substantially eliminated.

5. The method of claim 1, wherein the cell is selected from the group consisting of a CHO-K1 cell, a COS-7 cell, and a HEK293 cell.

6. The method of claim 1, wherein the agent is selected from the group consisting of a small molecule, a polypeptide, an antibody, a lipid, a polysaccharide, and a polynucleotide.

7. The method of claim 1, wherein the control is a cell engineered to express a mutant PAR2 polypeptide.

8. The method of claim 7, wherein the mutant PAR2 polypeptide comprises an amino acid sequence with at least 95% identity to SEQ ID NO:55.

9. The method of claim 1, wherein the agent binds the signal peptide sequence of the PAR2 intracellularly to disrupt the signal peptide function.

10. The method of claim 1, wherein the agent binds an allosteric site on the PAR2, and wherein binding of the agent to the allosteric site disrupts the signal peptide function.

11. A method of identifying an agent that activates a protease-activated receptor 2 (PAR2) intracellularly, the method comprising:
a. providing a cell expressing the PAR2 on a surface of the cell, wherein the PAR2 comprises a signal peptide sequence;
b. contacting the cell with an agent;
c. contacting the cell with a protease and/or a peptide ligand or small molecule;
d. measuring a level of activation of PAR2 upon contacting the cell with the protease and/or peptide ligand, wherein a reduction in the level of activation of PAR2 as compared to a control indicates that the agent is capable of activating PAR2 intracellularly.

12. The method of claim 11, wherein PAR2 is endogenously expressed.

13. The method of claim 11, wherein PAR2 is exogenously expressed.

14. The method of claim 13, wherein endogenous PAR2 expression is substantially eliminated.

15. The method of claim 11, wherein the cell is selected from the group consisting of a CHO-K1 cell, a COS-7 cell, and a HEK293 cell.

16. The method of claim 11, wherein the agent is selected from the group consisting of a small molecule, a polypeptide, an antibody, a lipid, a polysaccharide, and a polynucleotide.

17. The method of claim 11, wherein the control is a cell engineered to express a mutant PAR2 polypeptide.

18. The method of claim 17, wherein the mutant PAR2 polypeptide comprises an amino acid sequence with at least 95% identity to SEQ ID NO:55.

19. The method of claim 11, wherein the agent binds the signal peptide sequence of the PAR2 intracellularly to disrupt the signal peptide function.

20. The method of claim 11, wherein the agent binds an allosteric site on the PAR2, and wherein binding of the agent to the allosteric site disrupts the signal peptide function.

21. The method of claim 11, wherein the protease is selected from the group consisting of trypsin, tryptase, factor Xa TF, factor VIIa, matriptase/MT-serine protease 1, cysteine proteinase (RgpB), dust mite proteinase Der p3, dust mite proteinase Der p9, furin, and thrombin.

22. The method of claim 11, wherein the peptide ligand comprises SLIGKV (SEQ ID NO:1), SLIGRL-NH₂ (SEQ ID NO:58), or 2-furoyl-LIGRL-NH2 (SEQ ID NO:59).

23. The method of claim 11, wherein the small molecule is GB110.

24. An isolated mutant PAR2 polypeptide comprising an amino acid sequence selected from the group consisting of SEQ ID NO:45, SEQ ID NO:51, SEQ ID NO:53, and SEQ ID NO:55.

25. An isolated polynucleotide encoding the mutant PAR2 polypeptide of claim claim 24.

26. A vector comprising the isolated polynucleotide of claim 25.

27. A host cell comprising the vector of claim 26.

28. A method of producing an isolated mutant PAR2 polypeptide, the method comprising culturing the host cell of claim 27 under conditions suitable for the expression of the mutant PAR2 polypeptide and recovering the mutant PAR2 polypeptide from the cell or culture.
